SEQUENCE LISTING (6 sequences)

SEQ ID NO: 1 - protein, 389 aa, synthetic construct
MNSYPIVLVHGFMGWGRNEVLGLKYWGGITDYEQELSSYGYTAYTATVGPVSSNWDRACELYAYIKGGTVDYGHAHSTQKGHSRYGRTYPGLYPEWGNLTTEGKVNKIHLVAHSMGGQTVRTLVQLLKEGSEEERNTTPSQLSSLFAGGKSWVHSITTIASPHDGTTLADGINIFGDFAKNLVASLASFTGAGEKLIYDFKLDQWGLNRKSGESLTDYTNRVFNSAIWNSTNDLANWDLSTDGARVLNQWVKAQSDIYYFSYSTCATVPSILTSNELPHVIYMTPLLYPFGRFIGSYTRNEQGRVIIDNSWKPNDGVVNTISQNGPKIWSSDKIVNYNGVPQIGKWNSMPLLDTIDHMDACGIGTNALTLSWYKGLAEKLSQLTISNLE

SEQ ID NO: 2 - protein, 389 aa, synthetic construct
MNSYPIVLVHGFMGWGRNEVLGLKYWGGITDYEQELSSYGYTAYTATVGPVSSNADRACELYAYIKGGTVDYGHAHSTQKGHSRYGRTYPGLYPEWGNLTTEGKVNKIHLVAHSMGGQTVRTLVQLLKEGSEEERNTTPSQLSSLFAGGKSWVHSITTIASPHDGTTLADGINIFGDFAKNLVASLASFTGAGEKLIYDFKLDQWGLNRKSGESLTDYTNRVFNSAIWNSTNDLANWDLSTDGARVLNQWVKAQSDIYYFSYSTCATVPSILTSNELPHVIYMTPLLYPFGRFIGSYTRNEQGRVIIDNSWKPNDGVVNTISQNGPKIWSSDKIVNYNGVPQIGKWNSMPLLDTIDHMDACGIGTNALTLSWYKGLAEKLSQLTISNLE

SEQ ID NO: 3 - DNA, 1170 bp, synthetic construct
  source 1..1170: mol_type = other DNA
atgaacagttatcctatcgttttggtacacgggttcatgggttggggtcgtaacgaggtccttggattgaagtactggggcggtatcacggactacgagcaagagcttagttcgtacgggtacacggcctacacggcaactgtagggcccgtttcatctaactgggaccgtgcatgtgagttatacgcctacataaagggtggtacagttgactacggtcacgcacactccacacaaaagggtcactccagatacggtagaacataccccgggttgtaccccgagtggggtaacttaactacagaggggaaggtcaacaagatccacttagttgcacactccatgggtggtcaaacagttcgtacactcgttcaactgcttaaagaggggagtgaagaggagcgtaacactacaccctcacaactttcgtctttattcgccggtgggaagtcttgggttcacagtatcacaacgatcgcttcgcctcacgacggtacaacgttagcagacgggatcaacatcttcggagacttcgcaaagaaccttgttgcctctttagcatcgttcactggggcaggtgagaagttgatatacgacttcaagcttgaccaatggggtttaaacagaaagtcgggtgagtcacttacggactacacgaaccgtgtattcaactctgccatctggaactcaacgaacgacttggctaactgggacttatcaacagacggggctcgtgtccttaaccaatgggttaaggctcaaagtgacatctactacttctcatactccacatgtgccacggtcccatcaatattgacttctaacgagttgccacacgttatctacatgactcccctcttataccctttcgggagattcataggtagttacacacggaacgagcaaggaagagttataatcgacaactcttggaagcccaacgacggagttgtaaacacgataagtcaaaacggtccaaagatctggtcttcagacaagatcgtcaactacaacggtgttccccaaatcggtaagtggaacagtatgcccttacttgacactatcgaccatatggacgcatgtggtatcgggacgaacgcattaacgttgtcttggtacaagggtttagcagagaagctttcgcaattgacgataagtaacctcgagtga

SEQ ID NO: 4 - DNA, 1170 bp, synthetic construct; identical to SEQ ID NO: 3
  source 1..1170: mol_type = other DNA

SEQ ID NO: 5 - DNA, 45 bp, synthetic construct
  source 1..45: mol_type = other DNA
agcaaatgggtcgcggatccatgaacagttatcctatcgttttgg

SEQ ID NO: 6 - DNA, 42 bp, synthetic construct
  source 1..42: mol_type = other DNA
tggtggtggtggtgctcgagtcactcgaggttacttatcgtc

(19) United States
(12) Patent Application Publication
(10) Pub. No.: US 2026/0015596 A1
(43) Pub. Date: Jan. 15, 2026

(54) ENZYME WITH DEGRADATION ACTIVITY FOR HIGHLY RECALCITRANT PLASTIC AND METHOD OF BIODEGRADING HIGHLY RECALCITRANT PLASTIC USING THE SAME

(71) Applicant: KOREA INSTITUTE OF SCIENCE AND TECHNOLOGY, Seoul (KR)

(72) Inventors: Jung Ho AHN, Seoul (KR); Ga Hyun LEE, Seoul (KR); Do-Wook KIM, Seoul (KR); Gyeongtaek GONG, Seoul (KR); Ja Kyong KO, Seoul (KR); Youngsoon UM, Seoul (KR); Changmin SUNG, Seoul (KR); Eui Seok LIM, Seoul (KR)

(21) Appl. No.: 18/983,180

(22) Filed: Dec. 16, 2024

(30) Foreign Application Priority Data
Jul. 10, 2024 (KR) 10-2024-0090942

(51) Int. Cl. C12N 9/20; B09B 3/60; B09B 101/75; B09B 101/77

(52) U.S. Cl. CPC C12N 9/20; B09B 3/60; C12Y 301/01003; B09B 2101/75; B09B 2101/77

(57) ABSTRACT

Disclosed herein are an enzyme capable of degrading a highly recalcitrant plastic or a variant thereof, a composition comprising the same, and a method of degrading a highly recalcitrant plastic using the same. In addition, according to the present disclosure, polyethylene (PE) as a highly recalcitrant plastic can be effectively degraded as well as oxidized PE, which can be useful for developing plastic waste treatment and upcycling of plastic waste.

### CROSS REFERENCE TO RELATED APPLICATION

The present application claims priority to Korean Patent Application No. 10-2024-0090942, filed on Jul. 10, 2024, the entire contents of which are incorporated herein for all purposes by this reference.

### BACKGROUND OF THE INVENTION

### Field of the Invention

The present specification discloses an enzyme having plastic degradation activity.

### DESCRIPTION OF GOVERNMENT-SPONSORED RESEARCH

This study was conducted through the following national projects.
Department name: Ministry of Science and ICTc Project management (specialized) organization: Korea Institute of Science and Technology, Research project name: Support for research operation expenses of Korea Institute of Science and Technology (major project expenses), Research subject name: Development of Electro Super Cellulose Composite Material Source Technology, Project number: 2E33290
Department name: Ministry of Science and ICTc Project management (specialized) organization: Korea Research Foundation, Research project name: Development of Technology for Reducing Waste Plastics Using Radiation, Research project name: Development of Artificial Microorganisms for Customized PE Decomposition Based on Radiation, Project number: 00156234, Project unique number: 1711179272
Department name: Ministry of Science and ICTc Project management (specialized) organization: National Institute of Science and Technology Research, Research project name: Support for research operation expenses of the National Research Council of Science and Technology (major project expenses), Research project name: Microplastics generation and pollution reduction technology, Project number: CAP20024-300, Project unique number: 1711201840

### Description of the Related Art

Every year, 400 million tons of plastic products are produced, half of which are disposables and discarded within a year. Most plastics are disposed of by landfill and incineration, but most landfills have reached a saturated state and serious environmental pollution is caused in the process of landfilling and incineration. Due to heterogeneous plastics, non-plastic materials, other additives, or the like added to improve the properties of plastics, the recycling of plastic waste is limited, and because of issues such as the complex sorting and cleaning processes required in the recycling process and the degradation of physical properties with repeated physical recycling, currently only 9% of the world's plastic waste is recycled, 79% is landfilled, and 12% is incinerated.

Accordingly, although the demand for developing efficient plastic treatment methods is increasing, plastic products made of polyethylene (PE), polypropylene (PP), and polyvinyl chloride (PVC), which account for 60% of all plastics, are classified as non-degradable, and therefore, with treatment methods such as chemical decomposition and pyrolysis, degradation and recycling are limited, so the development of new degradation methods is required.

Since biological degradation takes place at room temperature, the biological degradation is energy efficient compared to incineration taking place at a temperature of 850° C. or more and chemical degradation and thermal degradation taking place at a temperature of 300° C. or more, and eco-friendly with no air pollution because of no generation of toxic gases such as dioxins and furans that occur in incineration and thermal degradation.

It has been proven that with the development of engineered leaf-branch compost cutinase, which can efficiently degrade polyethylene terephthalate (PET) into terephthalate and ethylene glycol monomers, and the increasing number of PET biorecycling companies, biodegradation of plastic waste is no longer an unrealistic goal. Enzymatic degradation is eco-friendly as it works under mild conditions without the use of chemicals that adversely affect the environment, such as strong acids, bases, or peroxides, and plastic degradation with enzymes does not produce toxic byproducts such as dioxins, furans, or phosgene. In addition, enzymatic degradation may facilitate the breakdown of plastics into smaller biodegradable compounds that can be assimilated by microorganisms, ultimately leading to the complete degradation of plastic waste into harmless materials, and the degradation products may be upcycled by metabolically engineered microorganisms for the production of high value-added chemicals and materials. Finally, enzymatic degradation enables precise degradation and subsequent plastic recycling through selectivity for specific chemical bonds in the plastic.

As described above, the enzymatic degradation of plastic waste is a promising alternative to conventional plastic waste disposal methods and can offer many benefits to solid waste management, but the development of biodegradation technologies for other types of plastics, other than PETc is still underdeveloped. In particular, PE, the most widely produced plastic, accounting for 40% of the world's plastic production, has a very low degradation rate due to its robust structural stability and hydrophobicity arising from its carbon chain consisting of only C—C and C—H bonds, and no specific oxidizing and hydrolyzing enzymes have been reported that can degrade PE.

In addition, PE degrades through a two-step reaction of oxidation and degradation. Generally, since PE waste dumped in the ocean and soil is naturally oxidized through abiotic reactions initiated by environmental factors such as sunlight, oxygen, and water through the mechanism of photooxidation, the development of biological degradation methods for substantially oxidized PE is necessary to treat accumulated PE waste.

### SUMMARY OF THE INVENTION

The Sequence Listing created on Aug. 8, 2024 with a file size of 9.34 KB, and filed herewith in XML file format as the file entitled “Sequence Listing_457KCL0097US,” is hereby incorporated by reference in its entirety.

In one aspect, the present disclosure is directed to providing an enzyme capable of biologically degrading a highly recalcitrant plastic and a composition comprising the same.

In another aspect, the present disclosure is directed to providing a method of biologically degrading a highly recalcitrant plastic.

To solve the object, an embodiment of the present disclosure may provide a lipase consisting of an amino acid sequence of sequence number 1, or a variant thereof.

An embodiment of the present disclosure may provide an isolated gene encoding the lipase or a variant thereof, or an expression vector comprising the same.

An embodiment of the present disclosure may provide a host cell transformed with the expression vector, or a culture thereof.

In addition, an embodiment of the present disclosure may provide a composition for degrading a highly recalcitrant plastic that comprises, as an active ingredient, a Pelosinus fermentans-derived lipase, a variant thereof, a host cell transformed with a vector comprising a gene encoding the lipase or a variant thereof, or a culture of the host cell.

An embodiment of the present disclosure may provide a method of degrading a highly recalcitrant plastic, comprising a step of treating and reacting a Pelosinus fermentans-derived lipase, a variant thereof, a host cell transformed with a vector comprising a gene encoding the lipase or a variant thereof, or a culture of the host cell with a plastic to be degraded.

In one aspect, the present disclosure may provide an enzyme capable of degrading PE, PP, and PVC, which are the most widely produced plastics worldwide but are highly resistant to degradation, or a variant thereof. The PE, PP, and PVC discarded in the environment are oxidized by sunlight and oxygen, and the enzyme or a variant thereof according to the present disclosure can specifically cleave the ester bonds in the C—C carbon chain of the oxidized PE, thereby effectively degrading the oxidized PE. Therefore, the present disclosure may be useful for developing plastic waste treatment and upcycling of plastic waste.

### BRIEF DESCRIPTION OF THE DRAWINGS

FIG. 1 is a schematic view illustrating a degradation mechanism for a recalcitrant plastic and a process of identifying a lipolytic enzyme for the degradation, according to an embodiment of the present disclosure.

FIG. 2 illustrates a phylogenetic tree of lipolytic enzyme families, with accession numbers obtained from GenBank. A branch length corresponds to the number of amino acid substitutions per site, and branches are annotated with 1,000 bootstrap replication rates exceeding 50%. The candidate enzymes selected for cloning are shown in gray circles, and the enzymes successfully expressed and purified in the present disclosure are highlighted in blue circles.

FIG. 3A, FIG. 3B, FIG. 3C, and FIG. 3D illustrate the evaluation results of candidate lipolytic enzymes using para-nitrophenyl acetate (p-NPA) cleavage assay. FIG. 3A is the result of hydrolysis of 4 μM of p-NPA by cutinase to generate para-nitrophenol (p-NP), FIG. 3B by esterase, FIG. 3C by hydrolase, and FIG. 3D by lipase, with enzyme concentrations for each enzyme category in the range of 0.03 to 2 μM. The generation of the p-NP was detected by measuring absorbance at 405 nm, and an error bar represents standard deviation derived from three experiments. TCCut1: Thermobifida cellulosilytica cutinase 1; AFEstA: Archaeoglobus fulgidus DSM 4304 esterase A; CBEstA: Clostridium botulinum esterase A; CHestA: Clostridium hathewayi esterase A; SerE: Serratia spp. esterase; HVH: Halomonas venusta hydrolase; OchH: Ochrobactrum sp. hydrolase; AML: Amycolaptosis mediterranei U32 lipase; CALB: Candida antarctica lipase B; PFL1: Pelosinus fermentans lipase 1; RAL: Rhizopus arrhizus lipase; RML: Rhizomucor miehei lipase; TLL: Thermomyces lanuginosus lipase.

FIGS. 4A and 4B illustrate the results of hydrolyzing octyl octanoate, which mimics the oxidized PE structure, using candidate lipolytic enzymes to evaluate PE degradation capabilities. FIG. 4A illustrates an amount of octyl octanoate remaining after treatment with 2 μM enzyme or no treatment (a control, Ctrl) in 10 mM of octyl octanoate for 24 hours, as percentage, and an error bar represents standard deviation derived from three experiments. A significant difference compared to the control was determined using a one-way analysis of variance (***: p<0.001; *: p<0.05). FIG. 4B illustrates a total ion chromatogram detected by gas chromatography/mass spectrometry (GC/MS) after hydrolysis of octyl octanoate using candidate enzymes, with hydrolysis products comprising (1) octanol and (2) octanoic acid with (3) octyl octanoate as substrate, labeled accordingly. Ctrl: Control; CBEstA: Clostridium botulinum esterase A; OchH: Ochrobactrum sp. hydrolase; PFL1: Pelosinus. fermentans lipase 1; RML: Rhizomucor miehei lipase; TCCut1: Thermobifida cellulosilytica cutinase 1; TLL: Thermomyces lanuginosus lipase.

FIG. 5 illustrates Fourier-transform infrared spectroscopy (FTIR) spectra of PE films treated with various candidate lipolytic enzymes after oxidation. O-H (3100-3650 cm⁻¹), C═O (1620-1750 cm⁻¹), and C-O (1170-1270 cm⁻¹) stretch peaks of various films were shown separately and labeled as (1), (2), and (3), respectively. The films comprise original PE film (PE), oxidized PE film (7-irradiated PE film; oxidized PE), and PE film treated with Clostridium botulinum esterase A (CBEstA), Pelosinus fermentans lipase 1 (PFL1), and Thermobifida cellulosylitica cutinase 1 (TCCut1).

FIG. 6 is a view illustrating the configuration of a vector pET-28a: PFL1 comprising PFL1 gene, according to an embodiment of the present disclosure.

FIG. 7 is a view of the results of confirmingp-NPA cleavage ability of PFL1, according to an embodiment of the present disclosure.

FIG. 8A is a view of the result of confirming octyl octanoate cleavage ability of PFL1, according to an embodiment of the present disclosure.

FIG. 8B is a view illustrating the result of confirming dioctyl ether cleavage ability of PFL1, according to an embodiment of the present disclosure.

FIG. 8C is a view illustrating the result of confirming 7-Hexadecanone cleavage ability of PFL1, according to an embodiment of the present disclosure.

FIG. 9A, FIG. 9B, FIG. 9C, FIG. 9D, FIG. 9E, FIG. 9F and FIG. 9G illustrate changes in physical properties of PE films after biodegradation using PFL1. FIG. 9A illustrates the result of gel permeation chromatography (GPC) analysis comparing oxidized PE film and PFL1-treated PE film (PFL1). FIG. 9B illustrates the result of differential scanning calorimetry (DSC) analysis of oxidized PE film and PFL1-treated PE film. FIGS. 9C and 9D are scanning electron microscopy (SEM) images of oxidized PE films. FIGS. 9E and 9F are SEM images of PFL1-treated PE films. FIG. 9G illustrates the result of water contact angle (WCA) analysis of original PE film (PE), oxidized PE film (7-irradiated PE film; oxidized PE), and PFL1-treated PE film (PFL1), respectively, with standard deviation derived from three replicate experiments.

FIG. 10A, FIG. 10B, FIG. 10C, and FIG. 10D illustrate the 3D structure prediction and molecular docking simulation results of PFL1. FIG. 10A illustrates the crystal structures of a closed form of PFL1 (left; closed) and the 3D open structure of PFL1^{W70} (right; open) predicted using Robetta server, with the dual lid domain shown in cyan. FIG. 10B illustrates a binding pose (in cyan) between PFL1 and pentacosyl pentacosanoate ligand obtained through molecular docking simulations using Autodock Vina 1.2.0 software. FIG. 10C is an expanded image illustrating hydrophobic interaction between PFL1 and pentacosyl pentacosanoate, with amino acid residues within enzyme surface shown in magenta. FIG. 10D illustrates a distance measured between carbonyl carbon of pentacosyl pentacosanoate and hydroxyl oxygen of catalytic serine using PyMOL software, with amino acid residues constituting catalytic triad highlighted in green.

FIG. 11 illustrates FTIR spectra of original PP film (PP), oxidized PP film (γ-irradiated PP film; oxidized PP), and PP film, which is oxidized and then treated with PFL1 (PFL1) according to an embodiment of the present disclosure.

FIG. 12 illustrates FTIR spectra of original PVC film (PVC), oxidized PVC film (γ-irradiated PVC film; oxidized PVC), and PVC film, which is oxidized and then treated with PFL1 (PFL1) according to an embodiment of the present disclosure.

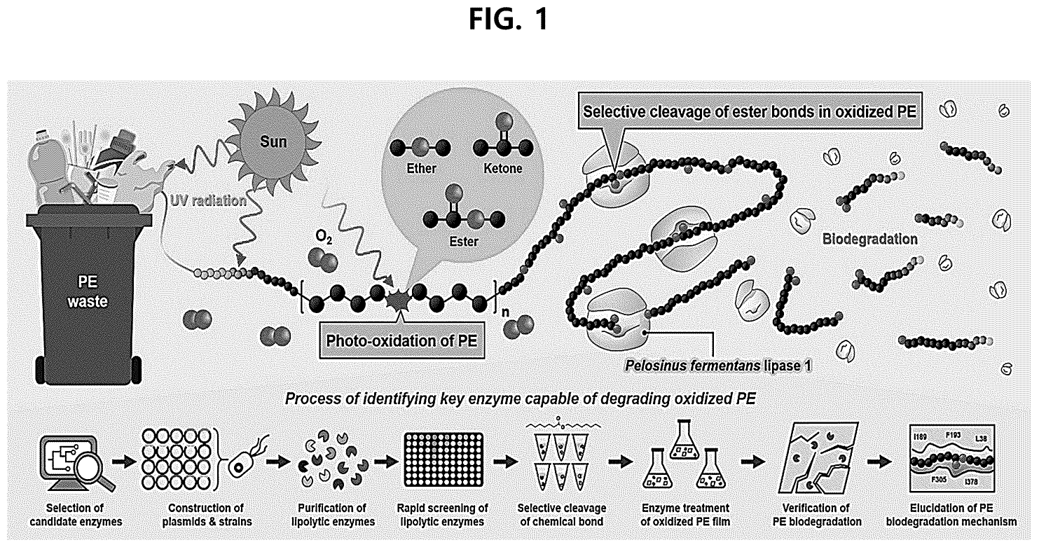

### DETAILED DESCRIPTION OF THE INVENTION

Hereinafter, exemplary embodiments of the present disclosure will be described in detail with reference to the accompanying drawings.

The embodiments of the present disclosure disclosed herein are illustrated for purposes of description only, and the embodiments of the present disclosure may be practiced in various forms and should not be interpreted as limiting to the embodiments described herein. The present disclosure is subject to various modifications and may have various forms, and the embodiments are not intended to limit the present disclosure to any particular disclosure form, but are to be understood to comprise all modifications, equivalents, or substitutions that fall within the scope of the spirit and art of the present disclosure. Singular expressions include plural expressions unless clearly described as different meanings in the context. In the present application, the terms “comprise,” “comprises,” “comprising,” “include,” “includes,” “including,” “contain,” “contains,” “containing,” “has,” “have,” “having” or other variations thereof are inclusive and therefore specify the presence of stated features, integers, steps, operations, elements, components, and/or combinations thereof, but do not exclude the presence or addition of one or more other features, integers, steps, operations, elements, components, and/or combinations thereof.

As used herein, the term “amino acid” refers to naturally occurring amino acids and synthetic amino acids, as well as amino acid analogs and amino acid mimetics that function in the same manner as naturally occurring amino acids. Naturally occurring amino acids may include those encoded by genetic codes, as well as those that are subsequently modified. Amino acid analogs may have the same basic chemical structure as naturally occurring amino acids, i.e., α carbon bonded to hydrogen, a carboxyl group, an amino group, and an R group, such as homoserine, norleucine, methionine sulfoxide, and methionine methyl sulfonium. These analogs may have a modified R group (e.g., norleucine) or a modified peptide backbone, but retain the same basic chemical structure as naturally occurring amino acids. Amino acid mimetics have a different structure than the typical chemical structure of amino acids, but function in a similar way to naturally occurring amino acids.

As used herein, amino acids may be referred to by the commonly known three-letter symbols or by one of the single-letter symbols recommended by IUPAC-IUB Biochemical Nomenclature Commission. Similarly, nucleotides may be referred to by the generally accepted single-molecule code.

As used herein, the term “nucleic acid” refers to deoxyribonucleotides or ribonucleotides, either in single- or double-stranded form, and polymers thereof. Unless specifically limited, the term includes nucleic acids that comprise, as reference nucleic acids, known analogs of naturally occurring nucleotides that have similar binding properties and are metabolized in a manner similar to naturally occurring nucleotides. Unless otherwise indicated, a specific nucleic acid sequence may implicitly comprise conservatively modified variants thereof and complementary sequences, as well as the sequence explicitly indicated.

As used herein, the term “lipase” may be used interchangeably with enzyme lipase, lipid hydrolase, or lipolytic enzyme that breaks down fats into fatty acids and monoglycerides. A “variant” of the lipase may comprise a non-naturally occurring sequence of amino acids. The variant has sequence identity or similarity of less than 100% to a wild-type amino acid sequence or nucleic acid sequence. For example, the variant may have sequence identity or similarity of less than about 75% to 100% to the amino acid sequence of the wild-type polypeptide throughout the length of the variant molecule. More specifically, the variant may have sequence identity or similarity of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%98%, or 99% or more. In an embodiment, the sequence identity or similarity may be defined as the percentage of amino acid residues that are identical to the amino acid residues of the wild type, after aligning the amino acid sequence of the variant and introducing gaps.

An embodiment of the present disclosure relates to an enzyme or a variant thereof capable of degrading a highly recalcitrant plastic. In an embodiment, the enzyme may be Pelosinus fermentans-derived lipase, and more particularly, the Pelosinus fermentans-derived lipase may be Pelosinus fermentans lipase 1 (PFL1).

In an embodiment, the lipase may be lipase consisting of the amino acid sequence of sequence number 1.

In an embodiment, the variant of the lipase may consist of the amino acid sequence of sequence number 2, in which T181-F204 and S229-N247 are modified in the amino acid sequence of sequence number 1. The amino acid sequence of sequence number 2 has sequence identity of 97% or more to sequence number 1, and may exhibit the same highly recalcitrant plastic resolution as lipase consisting of the amino acid sequence of sequence number 1.

The variant, according to an embodiment, is a variant with enhanced enzymatic activity of Pelosinus fermentans lipase, characterized in that the variant causes a structural change in alpha-helix 6 (T181 to F204) and alpha-helix 7 (S229 to N247) of the lipase, which may comprise a change in W70 residues.

The lipase or a variant thereof according to an embodiment may be manufactured in a transformed host cell using recombinant DNA technology. In this aspect, an embodiment of the present disclosure may provide an isolated gene sequence encoding the lipase or a variant thereof. In an embodiment, the lipase or a variant thereof may be encoded by a gene, more specifically a nucleic acid sequence.

In an embodiment, the isolated gene encoding the lipase or a variant thereof may consist of a nucleic acid sequence of sequence number 3.

In an embodiment, the isolated gene encoding a variant of the lipase may consist of a nucleic acid sequence of sequence number 4.

In an embodiment, the nucleic acid sequence encoding the lipase or a variant thereof may include, comprise, or essentially consist of the nucleic acid sequence of sequence number 3 or sequence number 4. In an embodiment, the nucleic acid sequence may comprise a nucleic acid sequence of 80%, 81%, 82%, 83%, 84%, 85%, 86%, 87%, 88%, 89%, 900%, 91%, 92%, 93%, 94%, 95%, 96%, 97%, 98%, or 99% or more identical to the nucleic acid sequence of sequence number 3 or sequence number 4.

An embodiment of the present disclosure may provide a vector capable of expressing the lipase or a variant thereof in a suitable host cell. In this aspect, an embodiment may provide an expression vector comprising a gene described above, specifically a recombinant expression vector.

As used herein, the “expression vector” is usually a recombinant carrier into which a fragment of heterologous DNA is inserted, generally referring to a fragment of double-stranded DNA. Here, heterologous DNA refers to foreign DNA, which is DNA that is not naturally found in a host cell. Once inside a host cell, an expression vector may replicate independently of host chromosomal DNA, resulting in the generation of a number of copies of the vector and inserted (heterologous) DNA thereof. In an embodiment, the expression vector may be one selected from the group consisting of plasmids, bacteriophages, viruses, cosmids, and artificial chromosomes, but is not limited thereto.

In an embodiment, the vector may comprise a nucleic acid sequence encoding the lipase or a variant thereof that operates by being linked to an appropriate expression regulatory sequence. In an embodiment, the vector may comprise a promoter operatively linked to the gene being cloned. As used herein, the “promoter” is one that promotes expression of the gene to be transfected, and the promoter may further comprise a basal element required for transcription, as well as an enhancer that may be used to promote and regulate expression.

In an embodiment, the expression vector may further comprise an expression cassette comprising a transcriptional promoter and a transcriptional termination site for the gene, in which the promoter and the termination site are operatively linked to the nucleic acid. In an embodiment, the expression cassette may further comprise, in addition to the promoter and termination signal site, an activator, an amplifier, an operator, a ribonucleases domain, start signals, cap signals, polyadenylation signals, and other signals that intervene in the regulation of transcription or translation.

In an embodiment, the expression vector may be used to transform a host cell, and the transformation may be performed using methods well known in the art.

In this aspect, an embodiment of the present disclosure may provide a host cell transformed with the expression vector. In addition, an embodiment of the present disclosure may provide a culture of the host cell.

In addition, an embodiment of the present disclosure may provide a method of manufacturing lipase or a variant thereof, which comprises steps of providing a host cell comprising a nucleic acid sequence encoding the lipase or a variant thereof, and maintaining the host cell under conditions in which the nucleic acid sequence encoding the lipase or a variant thereof is expressed.

As used herein, the term “transformation” refers to the introduction of the nucleic acid into a host cell such that the nucleic acid is replicable by an extrachromosomal factor or chromosomal integration.

In an embodiment, the type of host cell is not limited and may comprise, for example, interchangeability with an expression vector, toxicity of an enzyme encoded by a nucleic acid sequence, transformation rate, expression characteristics of the enzyme, biological safety, and costs. The balance of these factors should be based on the understanding that all host cells will not have the same effect on the expression of a specific nucleic acid sequence. Within general guidelines, the host cell may be a prokaryote or eukaryote in culture of a useful host cell. Specifically, the prokaryote may be a gram-positive or gram-negative bacterium. Specifically, the eukaryote may be a yeast or a fungus. More specifically, the host cell into which the expression vector is introduced may be Escherichia coli BL21(DE3).

In an embodiment, the transformed host cell may be cultured, or cultured and purified, thereby expressing lipase or a variant thereof for the purpose of the present disclosure. In an embodiment, the culturing and purification may each be performed by methods known in the art.

In an embodiment, the present disclosure may comprise steps of inducing protein expression while inoculating and culturing a transformed host cell in medium; collecting cells by centrifuging a culture upon completion of the culturing and then lysing the cells and centrifuging the lysates to separate a supernatant; and purifying the lipase or a variant thereof from the separate supernatant using a hexahistidine tag linked to a C-terminal or an N-terminal of the protein.

An embodiment of the present disclosure may provide a composition for degrading a highly recalcitrant plastic that comprises, as an active ingredient, Pelosinus fermentans-derived lipase, a variant thereof, a host cell transformed with a vector comprising a gene encoding the lipase or a variant thereof, or a culture of the host cell.

In addition, an embodiment of the present disclosure may provide a method of degrading a highly recalcitrant plastic, comprising a step of treating and reacting a plastic to be degraded with Pelosinus fermentans-derived lipase, a variant thereof, a host cell transformed with a vector comprising a gene encoding the lipase or a variant thereof, or a culture of the host cell.

An embodiment of the present disclosure may provide a use for Pelosinus fermentans-derived lipase, a variant thereof, a host cell transformed with a vector comprising a gene encoding the lipase or a variant thereof, or a culture of the host cell, for manufacturing a composition for degrading a highly recalcitrant plastic.

In an embodiment, the Pelosinus fermentans-derived lipase or a variant thereof, the host cell, or a culture of the host cell is as described above.

In an embodiment, the highly recalcitrant plastic may comprise one or more selected from PE, PP, and PVC. In an embodiment, the highly recalcitrant plastic may comprise one or more selected from oxidized PE, oxidized PP, and oxidized PVC.

Hereinafter, the present invention will be described in detail with reference to examples, comparative examples, and test examples. It will be apparent to those skilled in the art that the scope of the present invention is not limited by these examples, comparative examples, and test examples, which are being exemplarily presented solely to more specifically describe the present invention.

In this present disclosure, various enzymes of lipolytic enzyme families were investigated using a substrate that mimics oxidized PE to identify a key enzyme capable of degrading oxidized PE for the first time. In addition, detailed studies were conducted on the degradation of an oxidized PE film by a PE degrading enzyme to confirm and understand the degradation capabilities. Finally, structural analysis and molecular docking simulations of the PE degrading enzyme were performed to elucidate the PE degradation mechanism and obtain biochemical insights for future engineering efforts.

### [Test Example 1] Identification of Highly Recalcitrant Plastic Degrading Enzyme

### Phylogenetic Analysis

An amino acid sequence-based phylogenetic tree was configured using molecular evolutionary genetics analysis (MEGA) software version 11 using the maximum likelihood algorithm with 1,000 bootstrap samples. The phylogenetic tree comprises 32 enzymes across various lipolytic enzyme families. The amino acid sequences of these enzymes were provided by the national center for biotechnology information (NCBI) database.

### Bacterial Strains and Culture Media Composition

The strains, plasmids, and oligonucleotides used in the experiments are shown in Table 1 and Table 2 below.

TABLE 1

| Strain^{a} | Description^{b} |
|---|---|
| Escherichia coli | F⁻ φ 80lacZΔM15Δ(lacZYA-argF)U169 recA1 |
| DH5α | endA1 hsdR17(r_{K}⁻, m_{K}⁺) phoA supE44 λ⁻thi-1 |
| | gyrA96 relA1 |
| E. coli BL21(DE3) | F⁻ompT hsdS_{B} (r_{B}⁻, m_{B}⁻) gal dcm (DE3) |
| PE01 | E. coli BL21(DE3)::pETAML |
| PE02 | E. coli BL21(DE3)::pETAFC_N |
| PE03 | E. coli BL21(DE3)::pETAFC_C |
| PE04 | E. coli BL21(DE3)::pETAFEA |
| PE05 | E. coli BL21(DE3)::pETCALB_N |
| PE06 | E. coli BL21(DE3)::pETCALB_C |
| PE07 | E. coli BL21(DE3)::pETCCL_N |
| PE08 | E. coli BL21(DE3)::pETCCL_C |
| PE09 | E. coli BL21(DE3)::pETCRL_N |
| PE10 | E. coli BL21(DE3)::pETCRL_C |
| PE11 | E. coli BL21(DE3)::pETCBEA |
| PE12 | E. coli BL21(DE3)::pETCHEA |
| PE13 | E. coli BL21(DE3)::pETHH |
| PE14 | E. coli BL21(DE3)::pETOH_N |
| PE15 | E. coli BL21(DE3)::pETOT_C |
| PE16 | E. coli BL21(DE3)::pETPFL1 |
| PE17 | E. coli BL21(DE3)::pETPAL_N |
| PE18 | E. coli BL21(DE3)::pETPAL_C |
| PE19 | E. coli BL21(DE3)::pETPCL_N |
| PE20 | E. coli BL21(DE3)::pETPCL_C |
| PE21 | E. coli BL21(DE3)::pETPFL |
| PE22 | E. coli BL21(DE3)::pETRAL |
| PE23 | E. coli BL21(DE3)::pETRM |
| PE24 | E. coli BL21(DE3)::pETSE |
| PE25 | E. coli BL21(DE3)::pETTCC |
| PE26 | E. coli BL21(DE3)::pETTLL |
| PE27 | E. coli BL21(DE3)::pETUC_N |
| PE28 | E. coli BL21(DE3)::pETUC_C |

(^{a}N, Hexahistidine tag attached to N-terminal of encoded protein; C, Hexahistidine tag attached to C-terminal of encoded protein. ^{b}Kan^{R}, kanamycin resistance gene)

TABLE 2

| Plasmid^{a} | Description^{b} |
|---|---|
| pET-28a(+) | Expression vector; Kan^{R} |
| pETAML | pET-28a derivative containing gene encoding |
| | Amycolatopsis mediterranei U32 lipase (6.1 kb) |
| pETAFC_N | pET-28a derivative containing gene encoding |
| | Aspergillus fumigatus cutinase (5.9 kb) |
| pETAFC_C | pET-28a derivative containing gene encoding |
| | Aspergillus fumigatus cutinase (5.8 kb) |
| pETAFEA | pET-28a derivative containing gene encoding |
| | Archaeoglobus fulgidus DSM 4304 esterase A (6.3 kb) |
| pETCALB_N | pET-28a derivative containing gene encoding |
| | Candida antarctica lipase B (6.3 kb) |
| pETCALB_C | pET-28a derivative containing gene encoding |
| | Candida antarctica lipase B (6.2 kb) |
| pETCCL_N | pET-28a derivative containing gene encoding |
| | Candida cylindracea lipase (6.9 kb) |
| pETCCL_C | pET-28a derivative containing gene encoding |
| | Candida cylindracea lipase gene (6.8 kb) |
| pETCRL_N | pET-28a derivative containing gene encoding |
| | Candida rugosa lipase 1 (6.9 kb) |
| pETCRL_C | pET-28a derivative containing gene encoding |
| | Candida rugosa lipase 1 (6.8 kb) |
| pETCBEA | pET-28a derivative containing gene encoding |
| | Clostridium botulinum esterase A (6.7 kb) |
| pETCHEA | pET-28a derivative containing gene encoding |
| | Clostridium hathewayi esterase A (6.9 kb) |
| pETHVH | pET-28a derivative containing gene encoding |
| | Halomonas venusta hydrolase (6.4 kb) |
| pETOH_N | pET-28a derivative containing gene encoding |
| | Ochrobactrum sp. α/βhydrolase (6.4 kb) |
| pETOH_C | pET-28a derivative containing gene encoding |
| | Ochrobactrum sp. α/βhydrolase (6.3 kb) |
| pETPFL1 | pET-28a derivative containing gene encoding |
| | Pelosinus fermentans lipase 1 (6.5 kb) |
| pETPAL_N | pET-28a derivative containing gene encoding |
| | Pseudomonas aeuginosa lipase (6.2 kb) |
| pETPAL_C | pET-28a derivative containing gene encoding |
| | Pseudomonas aeuginosa lipase (6.1 kb) |
| pETPCL_N | pET-28a derivative containing gene encoding |
| | Pseudomonas cepacia lipase (6.4 kb) |
| pETPCL_C | pET-28a derivative containing gene encoding |
| | Pseudomonas cepacia lipase (6.3 kb) |
| pETPFL | pET-28a derivative containing gene encoding |
| | Pseudomonas fluorescens lipase (6.7 kb) |
| pETRAL | pET-28a derivative containing gene encoding |
| | Rhizopus arrhizus lipase (6.4 kb) |
| pETRM | pET-28a derivative containing gene encoding |
| | Rhizomucor miehei lipase (6.4 kb) |
| pETSE | pET-28a derivative containing gene encoding |
| | Serratia spp. esterase (6.0 kb) |
| pETTCC | pET-28a derivative containing gene encoding |
| | Thermobifida cellulosilytica cutinase (6.1 kb) |
| pETTLL | pET-28a derivative containing gene encoding |
| | Thermomyces lanuginosus lipase (6.1 kb) |
| pETUC_N | pET-28a derivative containing gene encoding |
| | uncultured organism carboesterase (6.6 kb) |
| pETUC_C | pET-28a derivative containing gene encoding |
| | uncultured organism carboesterase (6.5 kb) |

(^{a}N, Hexahistidine tag attached to N-terminal of encoded protein; C, Hexahistidine tag attached to C-terminal of encoded protein. ^{b}Kan^{R}, kanamycin resistance gene)

Escherichia coli (E. coli) DH5α and BL21 (DE3) strains were used as cloning hosts for genetic manipulation and protein production, respectively. Luria-Bertani (LB) medium was used in this experiment, and isopropyl β-D-thiogalactopyranoside (TPTG) and kanamycin were added when necessary to induce gene expression at final concentrations of 0.24 μg/L (1 mM) and 50 μg/mL, respectively.

### Manufacturing of Host Cell Comprising Hydrolytic Enzyme

The DNA sequence of a heterologous gene encoding an enzyme was obtained from NCBI database. The candidate genes were codon-optimized to ensure optimal expression within BL21(DE3) strain and chemically synthesized.

The synthesized gene fragment was introduced into pET-28a(+) degraded into BamHI and XhoI or NcoI and XhoI, and the hexahistidine tags were attached to the C- or N-terminal of the protein using Gibson Assembly Cloning kit (New England Biolabs, USA), respectively. A strain expressing the candidate enzymes was manufactured by transforming the BL21(DE3) strain with the recombinant plasmid.

The E. coli strains constructed above were cultured under conditions of 37° C. in LB medium (10 g NaCl, 10 g tryptone, 5 g yeast extract per liter) supplemented with 50 μg/mL of kanamycin until the optical density (0D₆₀₀) at 600 nm reached 0.6. After protein expression was induced with 1 mM of IPTG, cells were cultured for an additional 20 hours at 18° C. and harvested by centrifugation at 4,000 g for 15 minutes at 4° C. These cell pellets were resuspended in 50 mM Tris-HCl (tris(hydroxymethyl)aminomethane hydrochloride) at pH 8.0 and sonicated at 4° C. The lysed cells were centrifuged at 25,000 g for 20 minutes at 4° C. to remove cellular debris, and the supernatant was applied to a Nickel-nitrilotriacetic acid (Ni-NTA) agarose column. The proteins bound to the Ni-NTA synthetic resin was washed with Tris-HCl containing 15 to 30 mM of imidazole and purified by elution using Tris-HCl containing 300 mM of imidazole. The purity (>95%) of each protein was confirmed by sodium dodecyl sulfate-polyacrylamide gel electrophoresis (SDS-PAGE) analysis.

### Selection and Manufacturing of Lipase for PE Degradation

PE wastes disposed of in landfills or aquatic ecosystems typically undergo photooxidation when exposed to sunlight in the presence of oxygen or water, respectively. As a result, oxidized PE waste is hydrolyzed in the natural environment in the presence of water, and its efficiency may potentially be substantially improved with the help of enzymes. However, enzymatic hydrolysis of oxidized PE suffers from the limited accessibility of the enzyme to the oxidized PE surface.

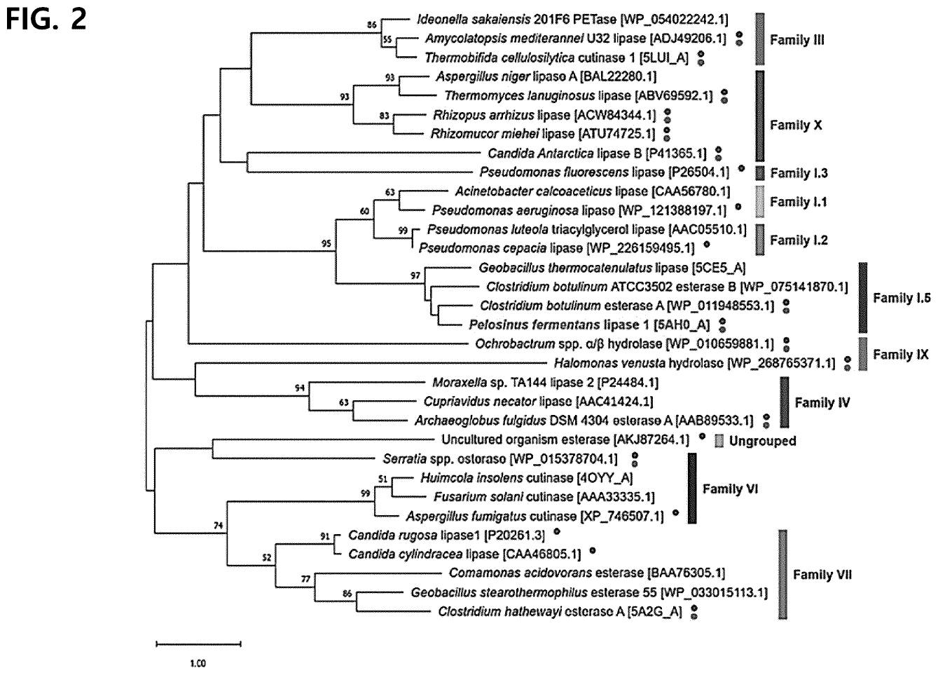

Among the hydrolytic enzymes identified so far, it is widely known that a lipolytic enzyme, which catalyzes the degradation of water-insoluble lipids, exhibits interfacial activity. To take advantage of these properties, a comprehensive investigation of various lipolytic enzymes, comprising esterase, lipase, cutinase, and other hydrolases, was conducted with the aim of identifying enzymes capable of degrading oxidized PE. In the present disclosure, 32 lipases belonging to different lipolytic enzyme families were investigated, and the interrelationships between these enzymes were revealed through the building of a phylogenetic tree (FIG. 2). While lipolytic enzymes share a common function in the hydrolysis of ester bonds, significant structural variation exists across the various families, and the preferred substrate types vary depending on the individual family.

From a pool of 32 lipolytic enzymes belonging to various families, 20 enzymes were selected and cloned for oxidized PE degradation (FIG. 2). Each enzyme underwent optimization process for expression in E. coli and was then analyzed using SDS-PAGE. However, expression of lipases from Pseudomonas and Candida species was not successful. As a result, 13 out of 20 lipolytic enzymes were successfully purified and these were directly used to identify enzymes capable of hydrolyzing oxidized PE.

### [Test Example 2] Lipolytic Enzyme Screening

### Lipolytic Enzyme Screening Using Para-Nitrophenyl Acetate Cleavage Assay

A simple and rapid p-NPA cleavage assay was used to screen candidate lipolytic enzymes for ester bond hydrolytic activity. Upon enzymatic hydrolysis, the colorless substrate, p-NPA, releases acetate and p-NP, which exhibits a yellow color due to absorbance at 405 nm. Therefore, the hydrolytic activity of 13 candidate enzymes toward p-NPA was evaluated by measuring changes in absorbance with enzyme concentration in the range of 0.03 to 2 μM. These enzymes were classified into four categories (cutinase, esterase, hydrolase, and lipase) based on their registered names in the NCBI database for easy comparison.

Specifically, the hydrolytic activity of candidate enzymes for PE degradation was evaluated by measuring absorbance at 405 nm using a microplate reader Spark (Tecan, Switzerland). The enzymatic reaction was carried out at 55° C. for 5 minutes using 50 mM of Tris-HCl (pH 8.0), 20% ethanol, 4 mM of p-NPA, 100 μL of 4 mM of reaction mixture, and candidate enzymes in the concentration range of 125 μM to 0.1 nM. Reaction mixtures without enzymes were denoted as blank.

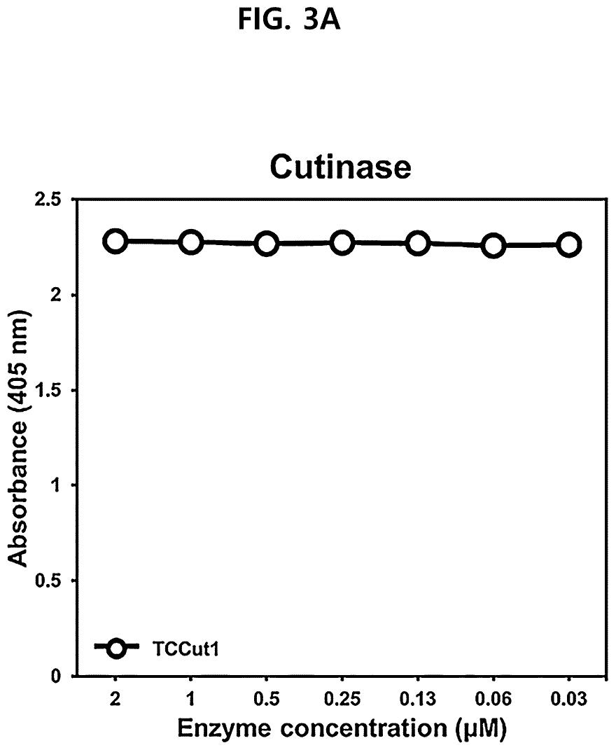

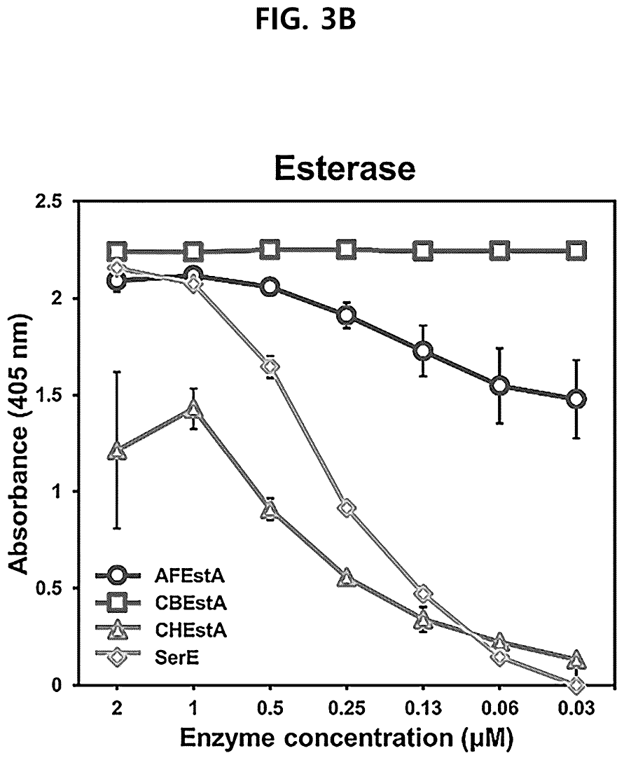

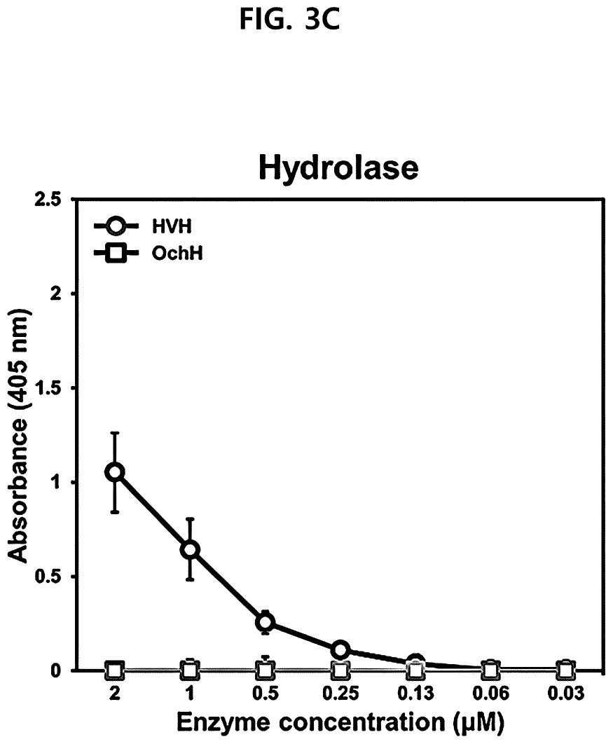

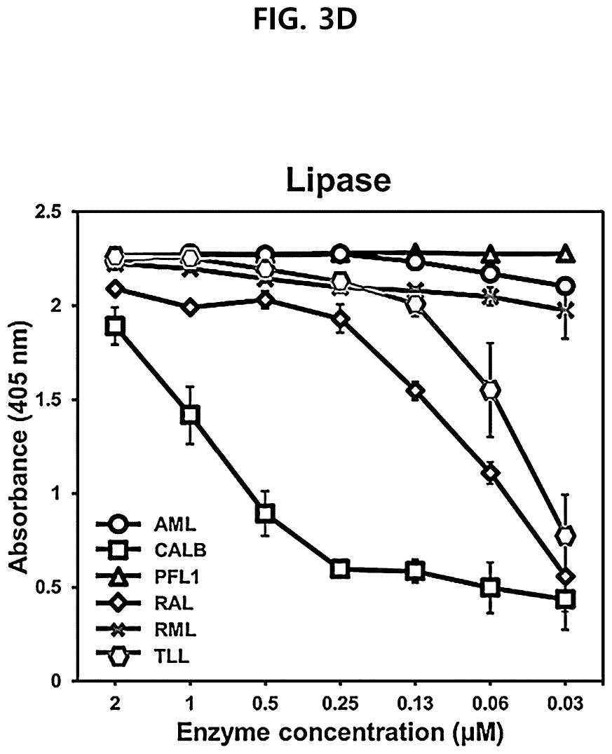

The results are shown in FIGS. 3A to 3D. Pelosinus fermentans lipase 1 (PFL1) according to an embodiment of the present disclosure, Clostridium botulinum esterase A (CBEstA), and Thermobifida cellulosilytica cutinase 1 (TCCut1), exhibited the highest hydrolytic activity (FIGS. 3A, B, and D). Further, the p-NPA cleavage assay performed using lower enzyme concentrations demonstrated the hydrolytic activity of PFL1, CBEstA, and TCCut1 even at enzyme concentration as low as 0.1 nM. PFL1 and CBEstA, which are members of the lipolytic enzyme family 1.5, have nearly identical protein structures (FIG. 2) and have been reported to degrade biodegradable plastics such as poly(butylene adipate-co-terephthalate) and poly(epsilon-caprolactone). Further, the lipolytic enzyme family III, to which TCCut1 belongs, is widely recognized for its proficiency in PET degradation. PFL1, CBEstA, and TCCut1 were potentially selected as degradation enzymes of oxidized PE on the basis of these findings and used in further studies described below.

### [Test Example 3] Identification of PE-Degrading Enzymes Using Compounds that Mimic Oxidized PE

To further evaluate the potential of PFL1, CBEstA, and TCCut1 in the degradation of oxidized PE, three types of 16-carbon chain compounds (octyl octanoate, dioctyl ether, and 7-hexadecanone) were used. These compounds mimic the ester, ether, and ketone functional groups expected to be present in the carbon chain of oxidized PE, respectively. To validate the results of the p-NPA cleavage assay, Rhizopus miehei lipase (RML), Thermomyces lanuginosus lipase (TLL), and Ochrobactrum sp. Hydrolase (OchH), which showed relatively high, medium, and low hydrolytic activity, respectively, were analyzed together with PFL1, CBEstA, and TCCut1.

### Cleavage Assay for Octyl Octanoate, Dioctyl Ether, and 7-Hexadecanone

After treatment with 2 μM of the enzyme for 24 hours, the hydrolytic activity of the candidate lipolytic enzymes was evaluated by quantifying the amount of degradation of 10 mM octyl octanoate, dioctyl ether, and 7-hexadecanone.

The enzymatic reaction was carried out in a 500 μL reaction mixture consisting of 50 mM Tris-HCl buffer (pH 8.0), 20% ethanol, 10 mM substrate, and 2 μM candidate enzyme for 24 hours at 30° C. To terminate the reaction above and extract analytes, 1 mL of chloroform was added, centrifuged at 21,130 g for 5 minutes to separate the chloroform layer from the enzyme waste, and gas chromatography/mass spectrometry (GC/MS) or gas chromatography (GC) was performed.

### GC/MS and GC Analysis

The degradation rate of octyl octanoate by the candidate enzymes was analyzed using an Agilent 7890B GC equipped with a 7010 triple quadrupole mass spectrometer (Agilent Technologies, USA). The separation of GC samples was performed on a DB-5MS capillary column (Agilent Technologies). The injector temperature was 230° C. and the ion source temperature was 250° C. Helium was used as a carrier gas at a constant flow rate of 1 mL/min. The injection volume was set to 1 μL and the split ratio was 10:1. The gradient heating program for the column was as follows: the initial oven temperature was 40° C. for 1 minute, then the temperature was increased to 240° C. at a rate of 10° C./min and held for 15 minutes. All compounds were quantified in multiple reaction monitoring mode by applying transition and collision energy voltages. Data analysis was performed using MassHunter qualitative analysis B.07.00 software (Agilent Technologies).

The analysis of the degradation of dioctyl ether and 7-hexadecanone was performed using an Agilent 6890N GC equipped with a flame ionization detector (Agilent Technologies) and an HP-INNOWax column. The gradient heating program of the column was as follows: the initial oven temperature was 100° C. for 5 minutes, followed by a temperature increase to 250° C. at a rate of 10° C./min, which was then held for 30 minutes. Data analysis was performed using ChemStation Rev B.04.03 software (Agilent Technologies).

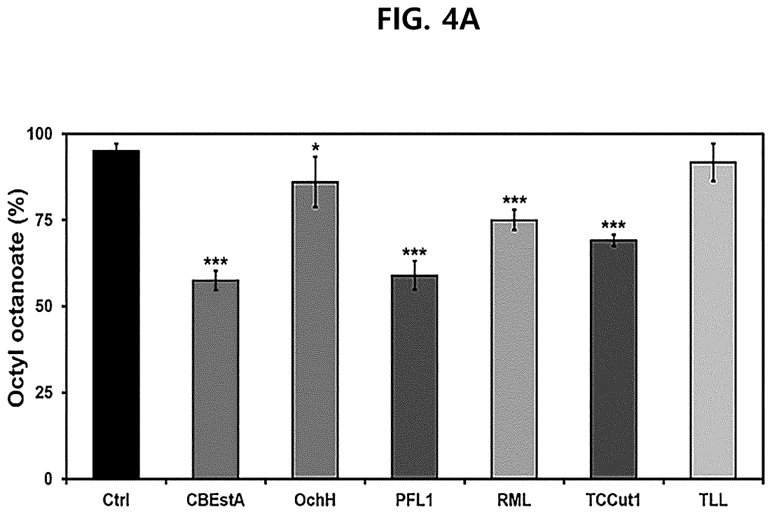

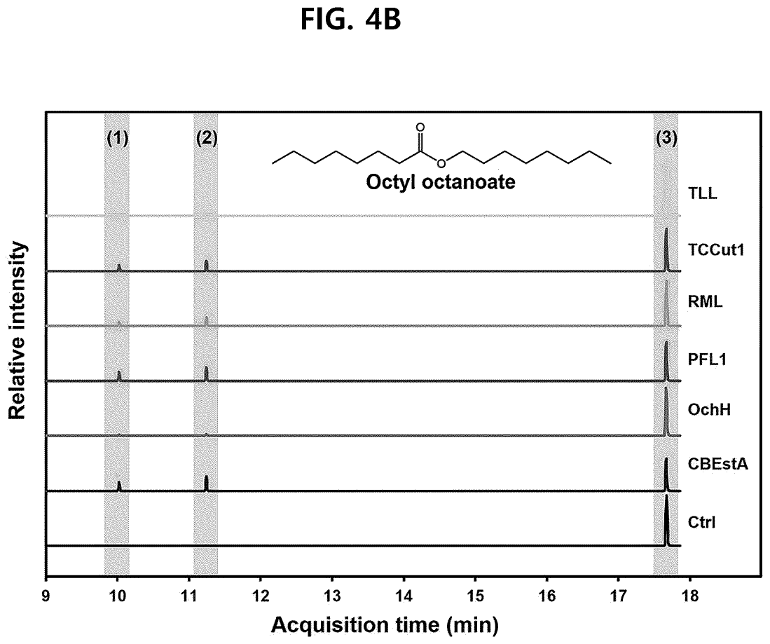

The results are shown in FIGS. 4A and 4B. All enzymes except TLL were found to be able to cleave octyl octanoate. Among these, PFL1, CBEstA, and TCCut1 exhibited the highest hydrolytic activity, cleaving 41, 42.5, and 31.7% of the provided octyl octanoate, respectively (FIG. 4A). Further, the production of octanol and octanoic acid as degradation products was observed from the cleavage of octyl octanoate (FIG. 4B). In contrast, hydrolysis of RML, TLL, and OchH exhibited limited degradation activity, consistent with the results of thep-NPA assay. In addition, all six candidate lipases tested were unable to hydrolyze dioctyl ether and 7-hexadecanone, demonstrating that these enzymes selectively target ester bonds within long carbon chain compounds.

### [Test Example 4] Enzymatic Degradation of Oxidized PE Film Using Lipolytic Enzymes

As the experiment above confirmed that PFL1, CBEstA, and TCCut1 effectively hydrolyze octyl octanoate, which is a substrate that mimics oxidized PE, these lipolytic enzymes were finally tested for the biodegradation of oxidized PE film.

### Enzymatic Treatment of Oxidized PE Film

To quickly simulate the oxidation process observed in PE waste exposed to outdoor environments (e.g., sunlight, oxygen, and water), a commercial PE film was exposed to 7-irradiation to obtain an oxidized PE film.

A roll of 20 m thick PE film (SK LiBS, South Korea) measuring 300×5.95 cm was placed in a 50 mL conical tube filled with distilled water. Subsequently, the conical tubes containing the PE film were irradiated at room temperature using the ⁶⁰Co-gammaa irradiator AECL IR-79 (MDS Nordion International Co., Ltd., Canada) of the Advanced Radiation Technology Institute (ARTI). The total absorbed dose administered was 300 kGy at a rate of 10 kGy/h.

Subsequently, the enzymatic degradation of the oxidized PE film was carried out at 30° C. and 200 rpm for 120 hours using the candidate enzymes. A 20 mL reaction mixture consisted of 50 mM Tris-HCl buffer at pH 8.0, a 1 cm×1 cm PE film sample, and 2 μM of the candidate enzyme. An additional 2 μM of enzyme was added to the reaction mixture every 48 hours to prevent loss of enzymatic activity. This resulted in a total of 6 μM of enzyme being used. The control sample was manufactured by culturing the oxidized film under the same conditions except that no enzyme was used. After enzymatic treatment, the film was subjected to a sequential cleaning process: initially, 2% (w/v) sodium dodecyl sulfate was used, followed by 20% ethanol. Finally, the enzyme-treated PE film sample was rinsed with distilled water and dried completely in a 50° C. oven for subsequent analysis. Reaction mixtures without candidate enzymes are denoted as blanks.

Fourier Transform Infrared Spectroscopy ofPE Film

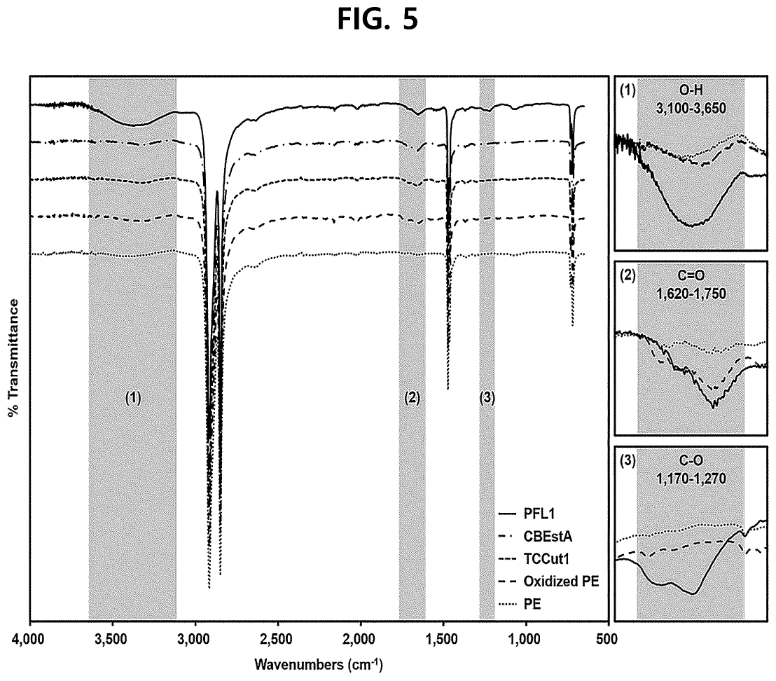

To identify the degradation of oxidized PFL1, CBEstA, and TCCut1, changes in the chemical composition of the enzyme-treated PE film were analyzed using FTIR (FIG. 5).

The FTIR on PE film was performed using a Nicolet iS10 spectrometer (Thermo Fisher Scientific, USA) equipped with an attenuated total reflectance sampling accessory featuring a diamond/ZnSe crystal plate. Each FTIR spectrum was recorded in the range of 4000 to 650 cm⁻¹ with a resolution of 4 cm⁻¹ using OMNIC software with an average of 64 scans per sample.

To demonstrate the effectiveness of the enzymatic treatment, the chemical composition of both the original unoxidized PE film and the oxidized PE film, as described above, was analyzed. The unoxidized PE film exhibited absorption peaks at 2910, 2850, 1472, 1463, 729, and 716 cm⁻¹, corresponding to bending and stretching vibrations related to aliphatic CH bonds. These peaks were equally observed in the oxidized PE film and the enzyme-treated PE film. Therefore, the peaks detected at the wavelength represent C—C and C—H bonds. Upon subsequent γ-irradiation treatment, an additional absorption peak appeared at 1620-1750 cm⁻¹, indicating the formation of C═O bonds from the oxidized PE film. Among the enzyme-treated PE films, significant changes were only observed in the PE film treated with PFL1. Absorption peaks at 1170-1270 cm⁻¹ (C—O stretch) and 3100-3650 cm⁻¹ (O—H stretch) appeared in the PE film treated with PFL1, indicating the hydrolysis of ester bonds in the oxidized PE film. In addition, there was no significant change in the absorption peak corresponding to the C═O stretch as PFL1 specifically cleaves the ester bond. These results suggest that the carbonyl groups formed on the PE film through radiation-induced oxidation undergo enzymatic hydrolysis to produce alcohols and carboxylic acids. Interestingly, CBEstA was unable to change the chemical composition of the oxidized PE film despite having the highest hydrolytic activity toward octyl octanoate (FIG. 4A).

### [Test Example 5] Manufacturing of Pelosinus fermentans-Derived Lipase

Pelosinus fermentans lipase 1 (Example 1), which is Pelosinus fermentans-derived lipase according to an embodiment of the present disclosure, was manufactured according to the method described in Test example 1.

Specifically, host cells were transformed by insertion of a vector containing the nucleic acid encoding Pelosinus fermentans lipase 1 below, from which Pelosinus fermentans-derived lipase (Example 1) was isolated and purified, respectively.

Sequence number 1 represents the amino acid sequence of the Pelosinus fermentans lipase 1 and sequence number 3 represents the nucleic acid sequence encoding the Pelosinus fermentans lipase 1.

Sequence number 2 represents the amino acid sequence of the variant, and sequence number 4 represents the nucleic acid sequence encoding the variant.

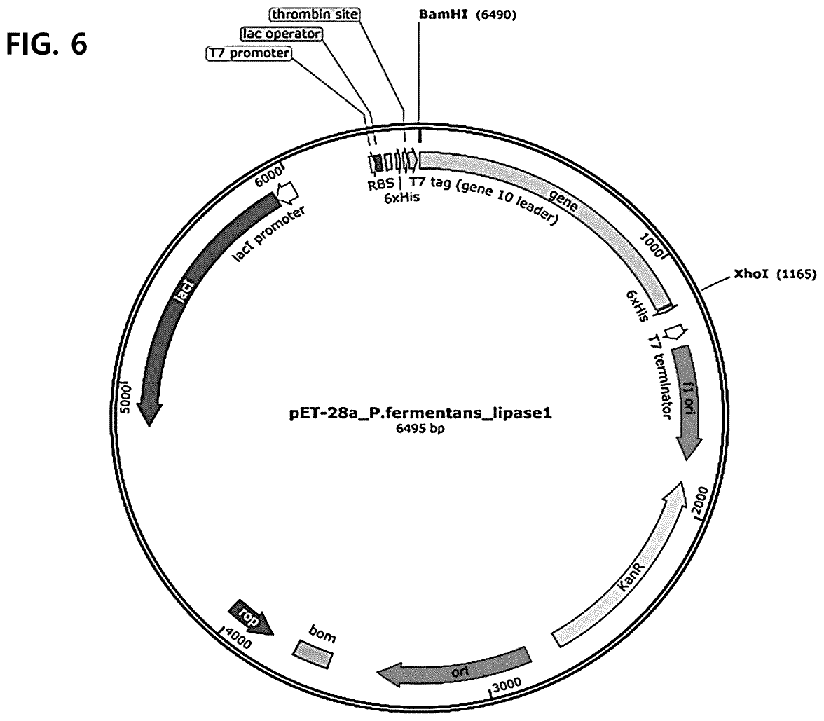

Sequence numbers 5 and 6 represent the primer sequences for the construction of the overexpression vector pET-28a:Pelosinus fermentans lipase 1. The overexpression vector pET-28a:Pelosinus fermentans lipase 1 was constructed by cloning a Pelosinus fermentans-derived lipase gene into the BamHI and XhoI sites of the pET-28a vector. The configuration of the vector pET-28a: Pelosinus fermentans lipase 1 containing the Pelosinus fermentans-derived lipase gene according to an embodiment of the present disclosure is illustrated in FIG. 6.

### [Test Example 6] Analysis of Degradation Performance Toward Small Molecule Substrates of Pelosinus fermentans Lipase 1

To confirm the degradation activity toward PE and oxidized PE of the Pelosinus fermentans lipase 1 of Example 1 manufactured in Test Example 5, the cleavage ability of p-NPA was analyzed in the same way as in Test Example 2. In addition, the cleavage ability of octyl octanoate, dioctyl ether, and 7-hexadecanone, which are three types of 16-carbon chain compounds comprised in oxidized PE, was analyzed using in the same way as in Test example 3.

In this case, the respective chemical structural formulas of octyl octanoate, dioctyl ether, and 7-hexadecanone are shown below. Among these, octyl octanoate comprises an ester bond, which breaks down into octanol and octanoic acid upon cleavage.

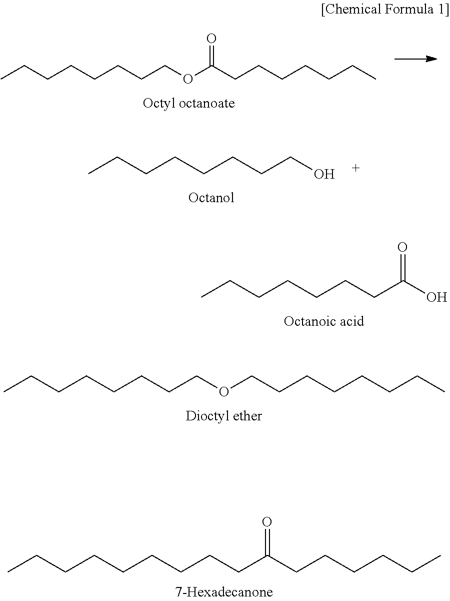

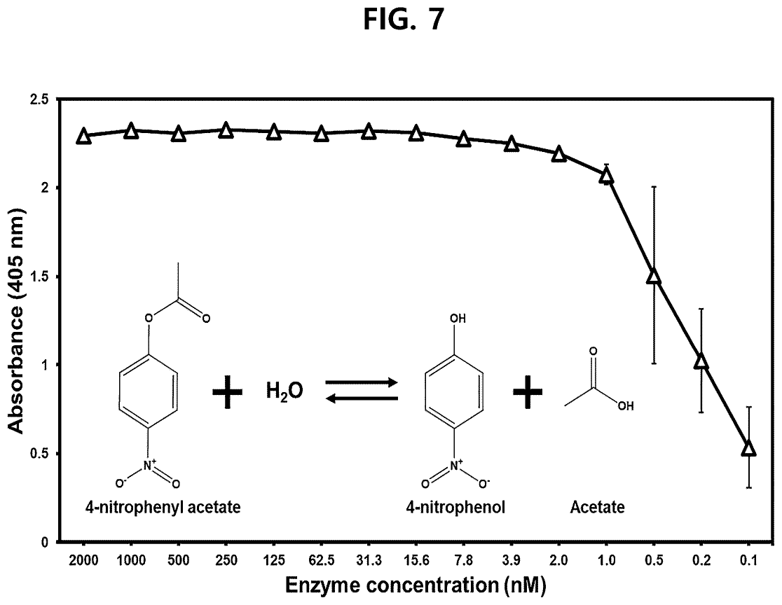

As a result, as illustrated in FIG. 7, when 4 mM of p-NPA was treated with 0.1 nM-2 μM of the Pelosinus fermentans lipase 1 of Example 1 above in conditions of 50 mM Tris-HCl, 50° C., 100% degradation of p-NPA occurred within 5 minutes. The degradation rate was measured with absorbance at 405 nm of p-NP, which is generated by cleavage of the ester bond of p-NPA.

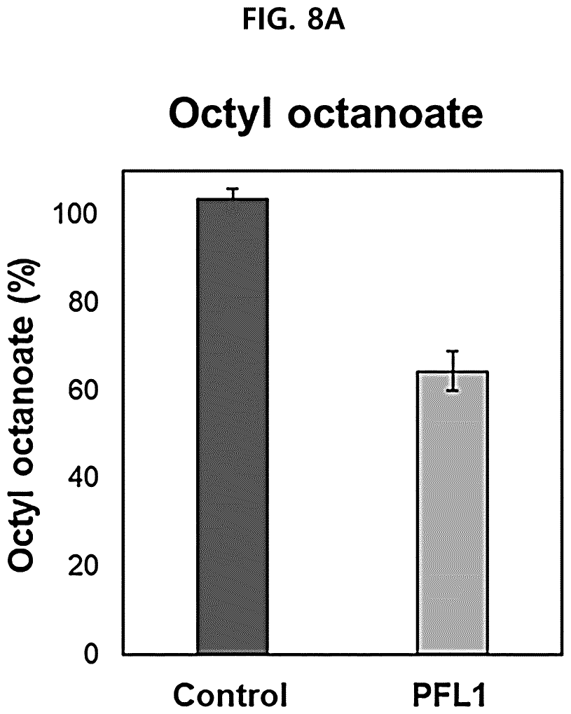

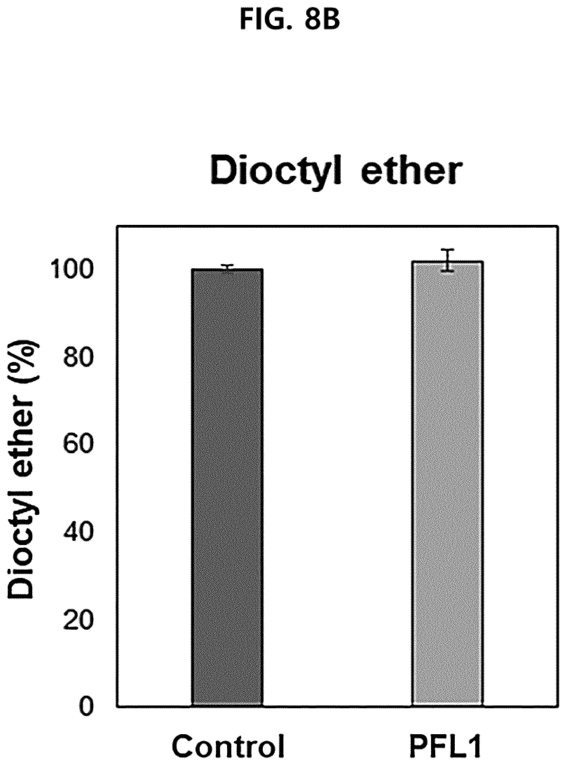

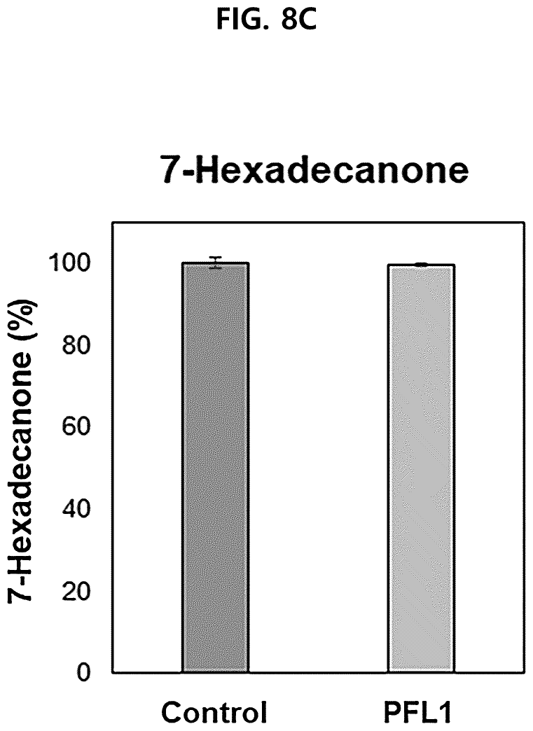

FIGS. 8A to 8C illustrate the results of the cleavage ability assays for octyl octanoate, dioctyl ether, and 7-hexadecanone, respectively. As illustrated in FIG. 8A, when octyl octanoate was treated with the Pelosinus fermentans lipase 1 of Example 1, 37.9% of octyl octanoate was degraded within 24 hours. The degradation rate was calculated as the residual amount of octyl octanoate after enzymatic treatment. As illustrated in FIGS. 8B and 8C, when dioctyl ether and 7-hexadecanone were treated with the Pelosinus fermentans lipase 1 of Example 1, respectively, neither dioctyl ether nor 7-hexadecanone was degraded within 24 hours. The degradation rate was calculated as the residual amount of dioctyl ether or 7-hexadecanone after enzymatic treatment. This means that Pelosinus fermentans lipase 1 according to an embodiment of the present disclosure is not only capable of cleaving long carbon chains, but also selectively cleaves only ester bonds and thus has degradation activity toward oxidized PE.

### [Test Example 7] Validation of Degradation of Oxidized PE Film by Pelosinus fermentans Lipase 1

The validation of the degradation of oxidized PE film by Pelosinus fermentans lipase 1 (PFL1) was performed by comprehensive analysis of the property changes of the PE film. First, the changes in Mₙ and M_{w} in the oxidized PE film after PFL1 treatment according to Example 1 above were analyzed using GPC.

### Gel Permeation Chromatoaphy of PE Film

The molecular weight of the PE film samples was analyzed at 160° C. using an EcoSEC HLC-8321 high temperature-gel permeation chromatography (HT-GPC) (Tosoh, Japan) equipped with a refractive index detector, a PLgel guard column, and two PLgel mixed-B columns. Prior to the analysis above, the PE film was dissolved in a solvent mixture of 1,2,4-trichlorobenzene and 0.04% butyl hydroxytoluene to adjust the concentration to 1.5 mg/mL, and 300 μL of the prepared sample was injected into the system at a column flow rate of 1.0 mL/min. The weight average molecular weight (M_{w}), number average molecular weight (Mₙ), and polydispersity index (PDI) were evaluated by analyzing refractive index chromatograms for polystyrene standards with known molecular weights.

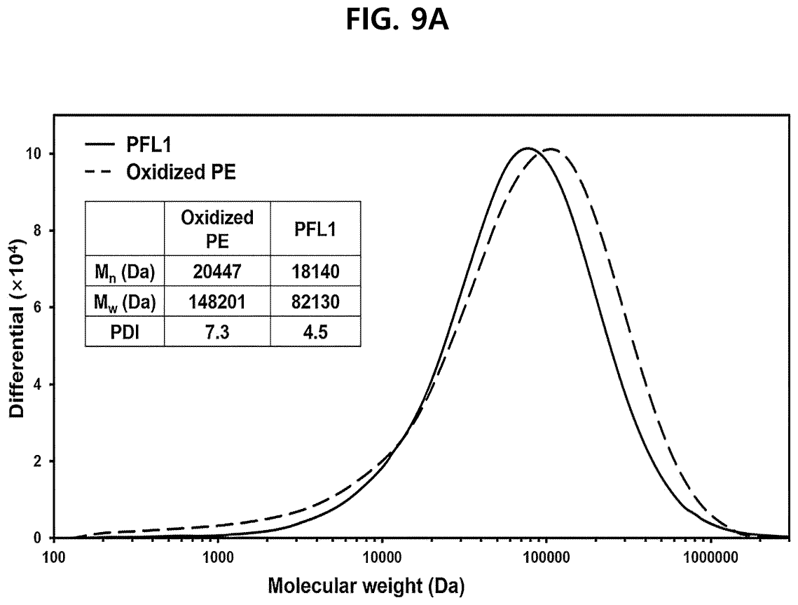

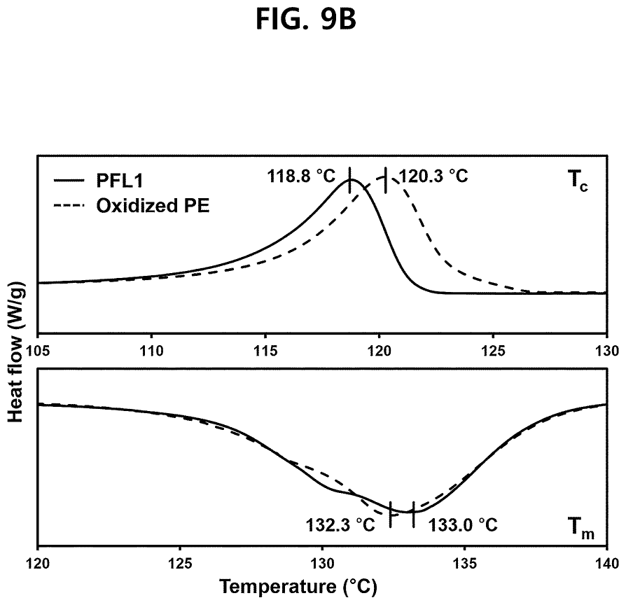

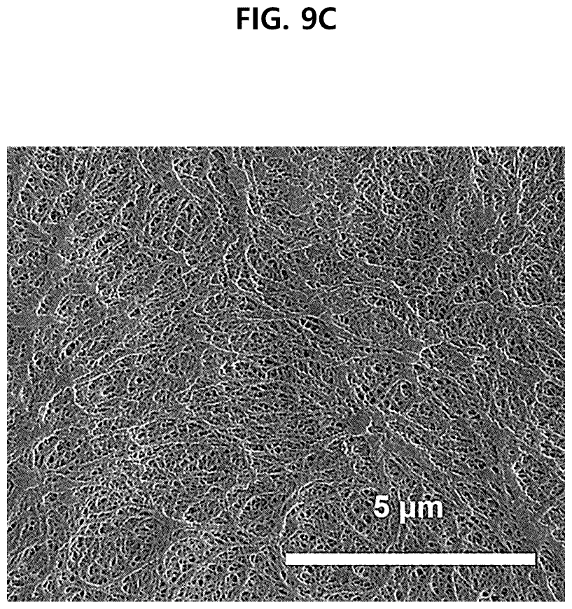

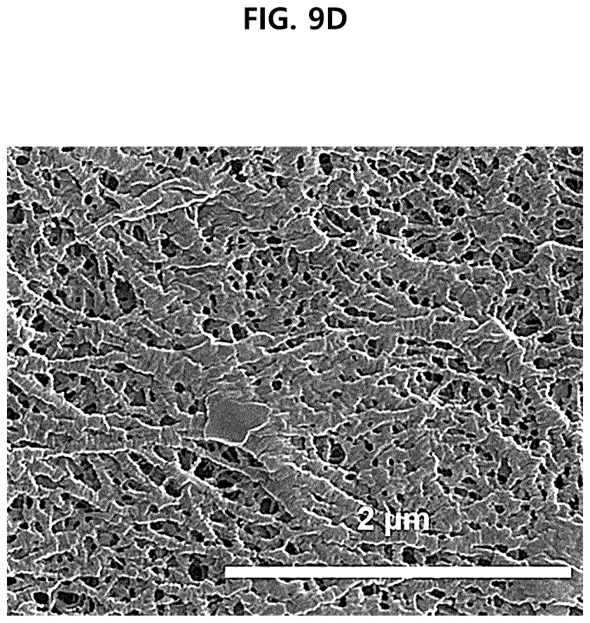

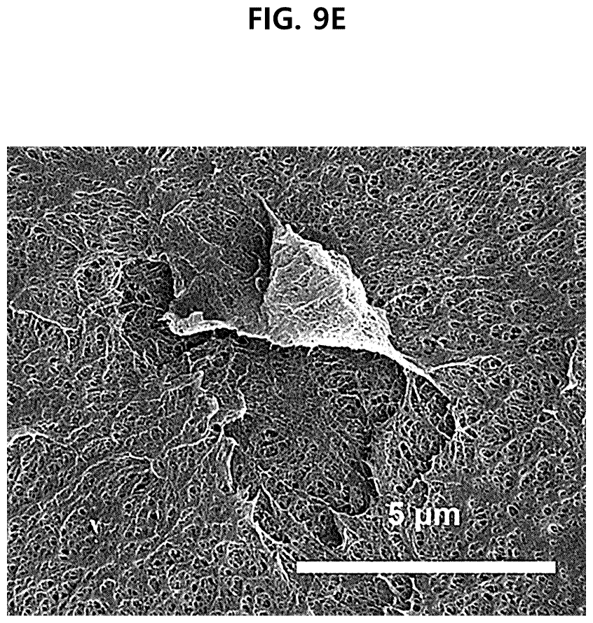

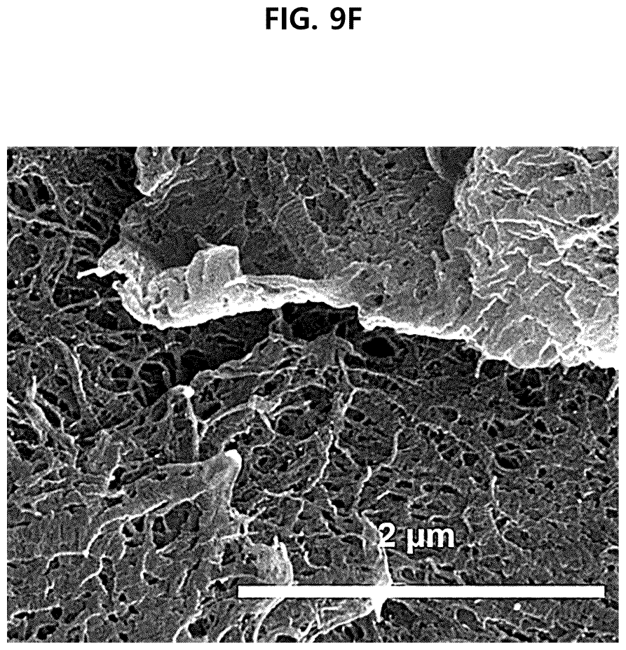

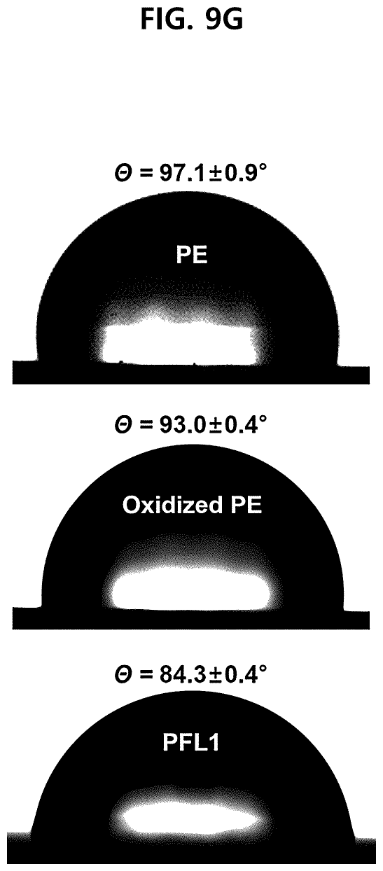

As illustrated in FIG. 9A, the Mₙ and M_{w} of the oxidized PE film treated with the PFL1 were significantly reduced to 18,140 Da and 82,130 Da, respectively, indicating a reduction of 11.3% and 44.6%, respectively, compared to the oxidized PE film above. Further, a significant reduction was observed in PE film treated with PFL1, particularly in the low molecular weight fraction.

### Differential Scanning Calorimetry of PE Film

To evaluate the changes in polymer crystallization caused by biodegradation, the changes in crystallization temperature T_{c} and melting temperature Tₘ of the oxidized PE film after PFL1 treatment in Example 1 above were analyzed using a DSC-25 differential scanning calorimetry (DSC) (TA instruments, USA).

The T_{c} and Tₘ values were analyzed by heating or cooling 5 mg of various PE films in the temperature range of 25 to 200° C. using a heating or cooling rate of 10° C./min.

As a result, as illustrated in FIG. 9B, the T_{c} value of the PFL1-treated PE film according to an embodiment of the present disclosure was found to be reduced to 118.81° C. compared to 120.25° C. of the oxidized PE film. This reduction in T_{c} value is believed to be due to PFL1 reducing the average molecular weight of the polymer, thus preventing the regular stacking of molten polymer chains. In contrast, the DSC analysis showed that the Tₘ value of the PFL1-treated PE film increased to 133.03° C. compared to 132.39° C. of the oxidized PE film, indicating that the proportion of the crystalline part of the polymer increased after the degradation of the polymer by PFL1. This is because of the higher susceptibility to enzymatic degradation due to the higher accessibility of amorphous regions compared to crystalline regions within the polymer. In addition, the DSC analysis showed melting peak splitting in the melting curves of both the oxidized PE film and the PFL1-treated PE film, indicating that the oxidation and PFL1 treatment resulted in the formation of inhomogeneous crystalline regions, respectively. Notably, the intensity of the melting peak splitting observed in the PFL1-treated PE film increased, suggesting that inhomogeneous enzymatic degradation occurred within the crystalline region.

### Scanning Electron Microscopy Measurement of PE Film

To visually evaluate the biodegradation of the oxidized PE film after PFL1 treatment, a scanning electron microscope (SEM) was used to examine the changes in surface morphology.

The surface morphology of the PE film sample was observed using a Teneo VolumeScope (Thermo Fisher Scientific). To prepare the sample for SEM analysis, the PE film sample was platinum coated for 1 minute and then subjected to SEM analysis at an accelerating voltage of 10 kV The SEM analysis comprises a magnification range of 30,000× to 100,000×.

As a result, the SEM micrographs of the oxidized PE film showed a smooth surface (FIG. 9C and FIG. 9D), while the photographs of the PFL1-treated PE film showed multi-layer degradation featuring surface erosion, numerous cracks, and deep tears (FIG. 9E and FIG. 9F). The surface morphology of the original PE film was almost identical to the surface morphology of the oxidized PE film. These findings strongly suggest that the changes in surface morphology observed in the PFL1-treated PE film are not due to autohydrolysis, but rather to effective hydrolysis by PFL1. In conclusion, this means that major highly recalcitrant plastic wastes may be treated in an eco-friendly manner using the biological degradation of plastics using PFL1.

### Analysis of Water Contact Angle of PE Film

The surface hydrophobicity changes of the oxidized PE film after PFL1 treatment in Example 1 above were analyzed using water contact angle (WCA) analysis.

The hydrophobicity changes of the PE film sample were evaluated using a Pheonix-10 contact angle analyzer (Surface & Electro Optics Co., Ltd, South Korea) equipped with Surfaceware 9 software (SEO Company) for image processing. To perform the WCA analysis, a completely dried PE film sample was placed on a glass slide, and then 4.5 μL of water was distributed to the sample using a micro-level syringe to measure a static contact angle θ formed by water droplets on the PE film surface.

As a result, the original PE film (PE in FIG. 9G) exhibits a contact angle of 97.07±0.94°, as illustrated in FIG. 9G, which is consistent with previous studies that a contact angle of 90° or more is indicative of a hydrophobic surface. The oxidized PE film (oxidized PE in FIG. 9G) exhibited a reduced contact angle of 93.04±0.44°, indicating a reduction in surface hydrophobicity due to the incorporation of oxygen-containing functional groups through γ-irradiation. Finally, the PFL1-treated PE film (PFL1 in FIG. 9G) exhibited a significantly reduced contact angle of 84.30±0.44°, indicating a further reduction in surface hydrophobicity due to effective hydrolysis by PFL1. These results are consistent with the formation of oxygen-containing functional groups in the PFL1-treated PE film, which was confirmed using FTIR (FIG. 5). In addition, the surface roughness of the oxidized PE film was improved, because enzymatic hydrolysis can affect the surface hydrophobicity. The identification of hydrolytic enzymes capable of degrading this oxidized PE and the biological degradation activity of PE using these hydrolytic enzymes is reported in the present disclosure for the first time.

Structural Analysis of Pelosinus fermentans Lipase 1 in PE Degradation

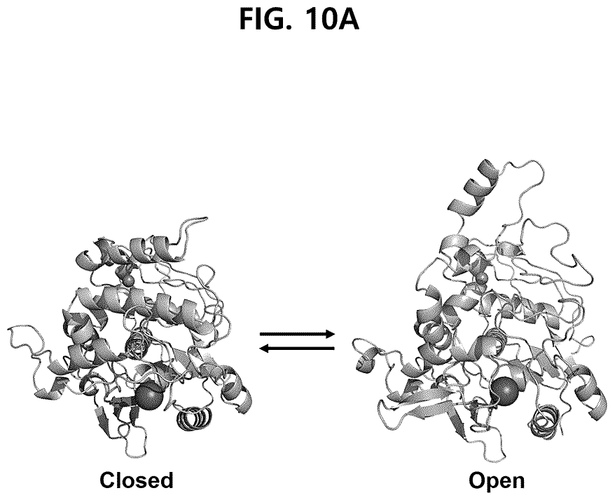

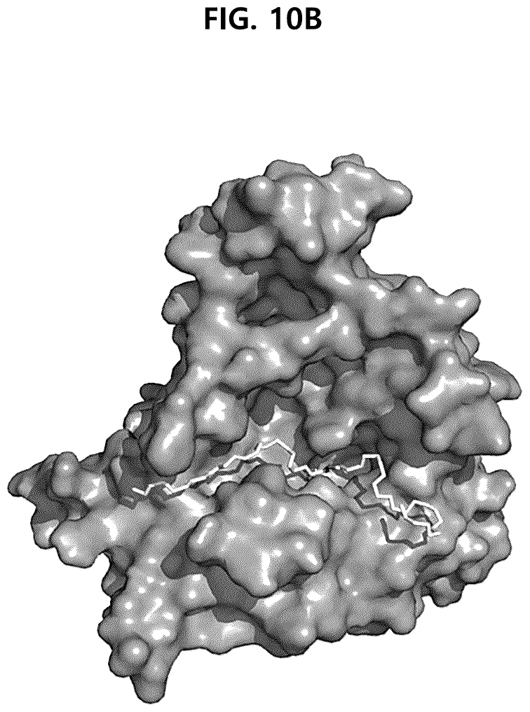

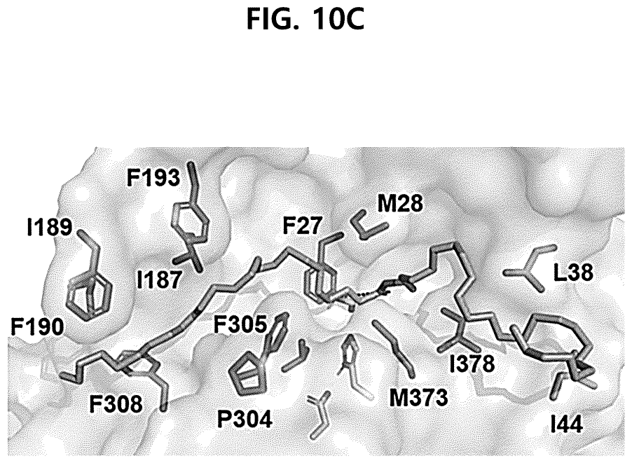

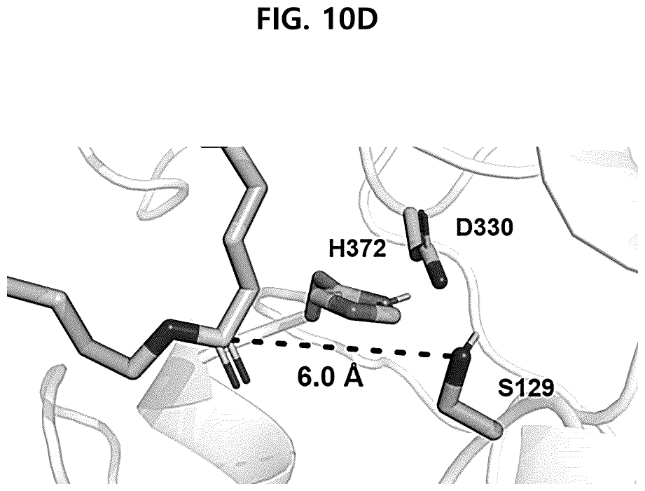

On the basis of the validation results of the biodegradation of oxidized PE using PFL1, the following experiment was performed to understand the unique properties of PFL1 that distinguish the PFL1 that degrades oxidized PE from other hydrolytic enzymes. The PFL1 shares similar structures with other lipases in lipolytic enzyme family 1.5, particularly a dual lid domain that surrounds numerous hydrophobic residues (FIG. 2 and FIG. 10A). This dual lid domain undergoes a conformational change from a closed state to an open state depending on the interaction between a hydrophobic residue and a substrate, allowing the substrate to access an active site. The dynamics of these structural changes are essential for the effective functioning of lipases in family I.5. CBEstA, another member of the lipolytic enzyme family 1.5, was proven to be unable to degrade oxidized PE film, despite exhibiting the highest hydrolytic activity toward octyl octanoate (FIG. 2, FIG. 3B, and FIG. 4A). This restriction was because of the presence of an additional loop comprising 71 amino acids at the N-terminal, which prevented the free movement of the dual lid domain. In contrast, PFL1 lacks an additional loop covering the dual lid domain, allowing the dual lid domain of PFL1 to move freely to promote PE degradation.

### Three-Dimensional Structure Prediction and Molecular Docking Simulation

Determining the PE degradation mechanism through molecular docking simulations requires a 3D open structure of PFL1, because PFL1 degrades oxidized PE only when this state is selected (FIG. 10A). However, the open crystal structure of PFL1 has not been reported. Members of lipolytic enzyme family 1.5 are known to possess a conserved tryptophan residue that is involved in conformational changes in the dual lid domain, and the crystal structure of the open structure of Bacillus thermocatenulatus lipase, which is another member of lipolytic enzyme family 1.5 (FIG. 2), was developed in another study by disrupting the conserved tryptophan in W61 (BTL2^{W61A}). With this knowledge, the 3D open structure of PFL1 was predicted using the Robetta server with the crystal structure of BTL2^{W61A} used as a template. The conformational change observed in BTL2^{W61A} was mimicked by disrupting the conserved tryptophan residue at W70 of PFL1 (PFL1^{W70A}). Structural comparison between the original crystal structure of PFL1 and the newly generated PFL1^{W70A} mutant model showed minimal distortion in the model, indicating that most of the PFL1 structure has been retained except for changes in the dual lid domain (FIG. 10A). Most importantly, according to the structural analysis, when PFL1 switches to the open conformation, numerous hydrophobic residues are exposed, which effectively promote the adsorption of substrate.

Then, the molecular docking simulation was performed using AutoDock Vina 1.2.0 for the newly developed PFL1^{W70A} mutant model. Taking into account the size of the exposed hydrophobic patch expected to interact with the PE on the PFL1^{W70A} mutant surface, pentacosyl pentacosanoate (C₅₀H₁₀₀O₂) was selected to simulate the long straight structure of the simultaneously oxidized PE, and an optimized molecular structure was constructed using Chem3D 22.2 (PerkinElmer Informatics Inc., USA) (FIG. 10B). The ligand molecules were docked to the PFL1^{W70A} mutant structure and generated up to 10,000 binding modes using Autodock Vina 1.2.0 software.

As a result, the pentacosyl pentacosanoate molecule was observed to form hydrophobic interactions with several hydrophobic residues comprising F27, M28, L38, 144, I187, I189, F190, F193, P304, F305, F308, M373, and 1378 on the PFL1^{W70A} mutant surface (FIG. 10C). Simultaneously, the ester group positioned at the center of the pentacosyl pentacosanoate molecule interacted with the catalytic serine positioned in the active site of the PFL1^{W70A} mutant (FIG. 10D). In several docking poses, a distance between the carbonyl carbon atom of the pentacosyl pentacosanoate molecule and the oxygen atom of the catalytic serine is in the range of 5 to 6 Å. This is consistent with the catalytic distances calculated for the active site of PET and PETase reported in previous studies, indicating that the docking simulation performed in the present disclosure accurately represents the actual binding of PFL1 to the substrate. In addition, a distance between the active site of PFL1 and the substrate is within an acceptable range for effective degradation of oxidized PE. In summary, the (1) large hydrophobic patch and (2) active site in close proximity to the ester group of oxidized PE allow PFL1 according to an embodiment of the present disclosure to effectively interact with long carbon chain PE, and these structural advantages mean that PFL1 can be used as an effective enzyme for degrading oxidized PE.

### [Test Example 8] Validation of Degradation of PP and PVC Film by Pelosinus fermentans Lipase 1

The following experiment was performed to identify whether the Pelosinus fermentans lipase 1 of the present disclosure also exhibits biodegradation of various other highly recalcitrant plastics besides PE.

Example 1 (PFL1) of the Pelosinus fermentans lipase 1 manufactured in Test example 5 above was used.

For highly recalcitrant plastics, PP and PVC were prepared. In this case, 25 m thick PP film (Sigma) and 200 m thick PVC film (Sigma) were prepared for the PP and PVC, respectively, which were γ-irradiated to prepare oxidized PP film and oxidized PVC film, respectively. The FTIR analysis to identify the enzymatic degradation of the oxidized PP film and oxidized PVC film and their degradation was performed under in the same way and conditions as in the Test example 4.

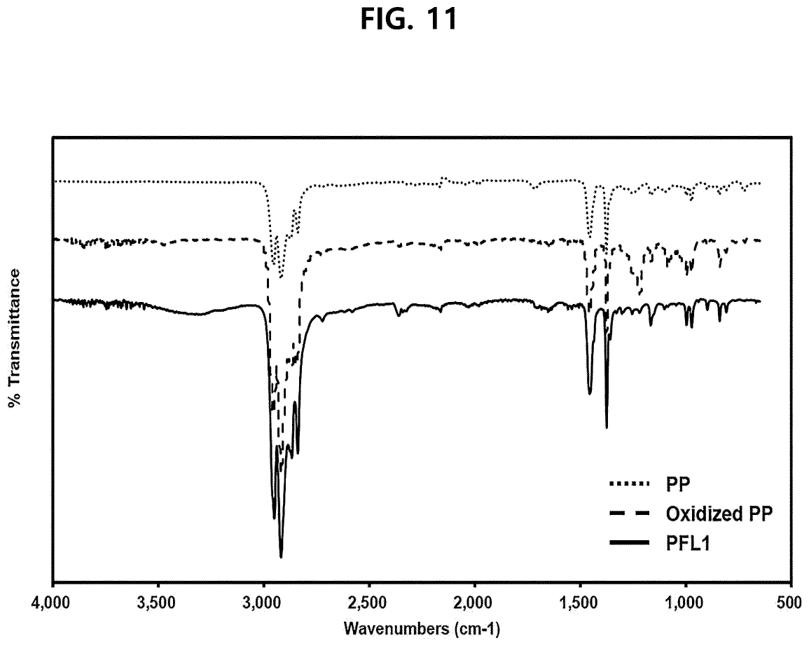

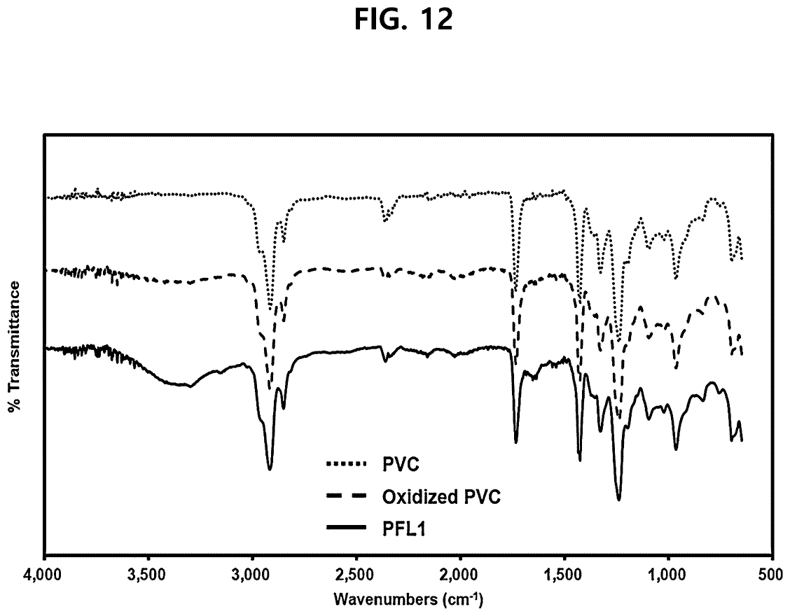

As a result, as in the case of enzymatic treatment on PE film, an O—H stretch at 3100 to 3650 cm⁻¹ was observed only when oxidized PP film was treated with PFL1 (FIG. 11). The oxidized PVC film was also observed to have a characteristic change in the O—H stretch at 3100 to 3650 cm⁻¹ only when treated with PFL1, with an increase in the C═O stretch at 1620 to 1750 cm⁻¹ (FIG. 12). Therefore, it can be seen that the Pelosinus fermentans lipase 1 according to the present disclosure exhibits biodegradability toward PP and PVC.

The present disclosure may provide the following embodiments as an example.

### First Embodiment

A lipase consisting of an amino acid sequence of sequence number 1 and a variant thereof.

### Second Embodiment

An isolated gene encoding the lipase or a variant thereof of the first embodiment.

### Third Embodiment

The gene of the second embodiment, wherein the isolated gene encoding the lipase consists of a nucleic acid sequence of sequence number 2.

### Fourth Embodiment

An expression vector comprising the gene of the second or third embodiment.

### Fifth Embodiment

The expression vector of the fourth embodiment, wherein the expression vector further comprises an expression cassette comprising a transcriptional promoter and a transcriptional termination site for the gene, and the promoter and the termination site are operatively linked to the nucleic acid.

### Sixth Embodiment

The expression vector of the fourth or fifth embodiment, wherein the expression vector is one selected from the group consisting of plasmids, bacteriophages, viruses, cosmids, and artificial chromosomes.

### Seventh Embodiment

A host cell transformed with a vector of any one of the fourth to sixth embodiments, or a culture thereof.

### Eighth Embodiment

The host cell of the seventh embodiment, wherein the host cell is a prokaryote or a eukaryote.

### Ninth Embodiment

The host cell of the seventh or eighth embodiment, wherein the prokaryote is a gram-positive or gram-negative bacterium.

### Tenth Embodiment

The host cell of the eighth or ninth embodiment, wherein the eukaryote is a yeast or a fungus.

### Eleventh Embodiment

A composition for degrading a highly recalcitrant plastic comprising: a Pelosinus fermentans-derived lipase, a variant thereof, a host cell transformed with a vector comprising a gene encoding the lipase or a variant thereof, or a culture of the host cell, as an active ingredient.

### Twelfth Embodiment

The composition of the eleventh embodiment, wherein the Pelosinus fermentans-derived lipase or a variant thereof is the lipase or a variant thereof of the first embodiment.

### Thirteenth Embodiment

The composition of the eleventh or twelfth embodiment, wherein the host cell, or a culture of the host cell, is the host cell, or a culture thereof, of any one of the seventh to tenth embodiments.

### Fourteenth Embodiment

The composition of any one of the eleventh to thirteenth embodiments, wherein the highly recalcitrant plastic comprises one or more selected from polyethylene, polypropylene, and polyvinyl chloride.

### Fifteenth Embodiment

The composition of any one of the eleventh to fourteenth embodiments, wherein the highly recalcitrant plastic comprises one or more selected from oxidized polyethylene, oxidized polypropylene, and oxidized polyvinyl chloride.

### Sixteenth Embodiment

A method of degrading a highly recalcitrant plastic, comprising: treating and reacting a Pelosinus fermentans-derived lipase, a variant thereof, a host cell transformed with a vector comprising a gene encoding the lipase or a variant thereof, or a culture of the host cell with a plastic to be degraded.

### Seventeenth Embodiment

The method of the sixteenth embodiment, wherein the Pelosinus fermentans-derived lipase or a variant thereof is the lipase or a variant thereof of the first embodiment.

### Eighteenth Embodiment

The method of the sixteenth or seventeenth embodiment, wherein the host cell, or a culture of the host cell, is the host cell, or a culture thereof, of any one of the seventh to tenth embodiments.

### Nineteenth Embodiment

The method of any one of the sixteenth to eighteenth embodiments, wherein the highly recalcitrant plastic comprises one or more selected from polyethylene, polypropylene, and polyvinyl chloride.

### Twentieth Embodiment

The method of any one of the sixteenth to nineteenth embodiments, wherein the highly recalcitrant plastic comprises one or more selected from oxidized polyethylene, oxidized polypropylene, and oxidized polyvinyl chloride.

### STATEMENT REGARDING PRIOR DISCLOSURE BY THE INVENTOR OR A JOINT INVENTOR

The inventors of the present application have made the following related disclosure: Do-Wook KIM et al., “Biodegradation of oxidized low density polyethylene by Pelosinus fermentans lipase,” Bioresource Technology, Vol. 403, No. 130871 on May 21, 2024. The related disclosure was made less than one year before the effective filing date (Jul. 10, 2024) of the present application. The authors of the related disclosure include four authors (Hyeoncheol Francis Son, Jong-Hyun Jung, Hyun June Park and Sung Ok Han) who are not named as the joint inventors of the present application. However, they are graduate students or technicians worked under the direction and supervision of the joint inventors and do not contribute to the conception of the invention, and thus these authors are not joint inventors of the present application. Accordingly, the related disclosure is disqualified as prior art under 35 U.S.C § 102(a)(1) against the present application. See 35 U.S.C § 102(b)(1)(A).

What is claimed is:

1. A lipase consisting of an amino acid sequence of sequence number 1 and a variant thereof.

2. An isolated gene encoding the lipase or a variant thereof of claim 1.

3. The gene of claim 2, wherein the isolated gene encoding the lipase consists of a nucleic acid sequence of sequence number 2.

4. An expression vector comprising the gene of claim 2.

5. The expression vector of claim 4, wherein the expression vector further comprises an expression cassette comprising a transcriptional promoter and a transcriptional termination site for the gene, and the promoter and the termination site are operatively linked to the nucleic acid.

6. The expression vector of claim 5, wherein the expression vector is one selected from the group consisting of plasmids, bacteriophages, viruses, cosmids, and artificial chromosomes.

7. A host cell transformed with the expression vector of claim 4, or a culture thereof.

8. The host cell of claim 7, wherein the host cell is a prokaryote or a eukaryote.

9. The host cell of claim 8, wherein the prokaryote is a gram-positive or gram-negative bacterium.

10. The host cell of claim 8, wherein the eukaryote is a yeast or a fungus.

11. A composition for degrading a highly recalcitrant plastic comprising:
a Pelosinus fermentans-derived lipase, a variant thereof, a host cell transformed with a vector comprising a gene encoding the lipase or a variant thereof, or a culture of the host cell, as an active ingredient.

12. The composition of claim 11, wherein the Pelosinus fermentans-derived lipase or a variant thereof is a lipase or a variant thereof consisting of an amino acid sequence of sequence number 1.

13. The composition of claim 11, wherein the host cell, or a culture of the host cell, is a host cell transformed with an expression vector comprising an isolated gene encoding a lipase consisting of an amino acid sequence of sequence number 1 or a variant thereof, or a culture of the host cell.

14. The composition of claim 11, wherein the highly recalcitrant plastic comprises one or more selected from polyethylene, polypropylene, and polyvinyl chloride.

15. The composition of claim 11, wherein the highly recalcitrant plastic comprises one or more selected from oxidized polyethylene, oxidized polypropylene, and oxidized polyvinyl chloride.
